SEQUENCE LISTING (16 sequences)

SEQ ID NO: 1 - DNA, 18 bp, mouse
attgggcagatcgtcacc

SEQ ID NO: 2 - DNA, 20 bp, mouse
acagcactcagcacgatcac

SEQ ID NO: 3 - DNA, 21 bp, mouse
gatgccttcctggatctcatt

SEQ ID NO: 4 - DNA, 23 bp, mouse
tctttgtcactgtctgaatctgc

SEQ ID NO: 5 - DNA, 20 bp, mouse
ccgctcgctaagctgttact

SEQ ID NO: 6 - DNA, 25 bp, mouse
ctttggtgttagagaggacaacttt

SEQ ID NO: 7 - DNA, 24 bp, rat
cagagcagccctaaggcactttcc

SEQ ID NO: 8 - DNA, 25 bp, rat
ccgatgacggtgccgatgatgatgg

SEQ ID NO: 9 - DNA, 21 bp, mouse
cctggcatttggacgctacat

SEQ ID NO: 10 - DNA, 22 bp, mouse
tgagaattgctgtgagcttgca

SEQ ID NO: 11 - DNA, 24 bp, rat
gccctatcaactttcgatggtagt

SEQ ID NO: 12 - DNA, 21 bp, rat
ggatgtggtagccgtttctca

SEQ ID NO: 13 - DNA, 20 bp, mouse
gtaacccgttgaaccccatt

SEQ ID NO: 14 - DNA, 20 bp, mouse
ccatccaatcggtagtagcg

SEQ ID NO: 15 - DNA, 20 bp, mouse
cttatagatccaaaaaatat

SEQ ID NO: 16 - DNA, 20 bp, mouse
aaatgaagaccgagtccttc

(19) United States
(12) Patent Application Publication
(10) Pub. No.: US 2016/0199393 A1
(43) Pub. Date: Jul. 14, 2016

(54) METHODS OF TREATING BRAIN ISCHEMIA OR HYPOXIA

(71) Applicant: China Medical University, Taichung City (TW)

(72) Inventors: Chia-Hung HSIEH, Taichung City (TW); Yu-Jung LIN, Taichung City (TW); Woei-Cherng SHYU, Taichung City (TW)

(21) Appl. No.: 14/829,604

(22) Filed: Aug. 18, 2015

Related U.S. Application Data
(60) Provisional application No. 62/101,338, filed on Jan. 8, 2015.

(51) Int. Cl. A61K 31/655; A61K 31/517; A61K 31/506; A61K 31/44

(52) U.S. Cl. CPC A61K 31/655; A61K 31/44; A61K 31/517; A61K 31/506

(57) ABSTRACT

Methods of treating brain ischema or hypoxia by using an inhibitor of cysteine-glutamate transporter (i.e. system x_{c}⁻) is provided. The inhibitor includes sorafenib and a derivative thereof, erastin, and suifasalazine. These inhibitors can effectively decrease a concentration of extracellular glutamate, so that excitotoxicity to central nervous system (CNS) and a cortical infarct volume in brains can be reduced.

### CROSS-REFERENCE TO RELATED APPLICATION

This application claims the priority benefit of U.S. provisional application Ser. No. 62/101,338, filed Jan. 8, 2015, the full disclosure of which is incorporated herein by reference.

### SEQUENCE LISTING

The sequence listing submitted via EFS, in compliance with 37 CFR §1.52(e)(5), is incorporated herein by reference. The sequence listing text file submitted via EFS contains the file “TWT04033US_SequenceListing”, created on Aug. 12, 2015, which is 2,449 bytes in size.

### BACKGROUND

1. Field of Invention

The disclosure relates to methods and compositions of treating brain ischemic or hypoxia.

2. Description of Related Art

Stroke is a leading cause of death and long-term disability in developed countries, and represents a major economic burden in the world (Dombovy M L, Sandok B A, Basford J R. Rehabilitation for stroke: a review. Stroke; a journal of cerebral circulation. 1986; 17(4363-9). Substantial evidence indicates that glutamate-mediated excitotoxicity is a major contributor to the resulting neuropathology in stroke victims (Rothman S M, Olney J W. Glutamate and the pathophysiology of hypoxic-ischemic brain damage. Annals of neurology. 1986; 19(2):105-11). However, to date, the development of effective clinical treatments for this potentially devastating condition has been largely unsuccessful, because it is difficult to inhibit simultaneouslyy the various glutamate receptors and their activated enzymes during a stroke (Lai T W, Shyu W C, Wang Y T. Stroke intervention pathways: NMDA receptors and beyond. Trends in molecular medicine. 2011; 17(5):266-75). Therefore, it is well accepted that inhibiting stroke-induced elevated extracellular glutamate is more effective than inhibiting all glutamate receptors for the prevention of excitotoxicity. However, there are no therapeutics available for this purpose.

It has been shown that hypoxia or ischemia-mediated reduction in adenosine triphosphate (ATP) causes failure of the energy-mediated function of Na⁺ pumps and leads to accumulation of Na⁺ ions inside neurons, contributing to cellular membrane depolarization and glutamate exocytosis (Choi D W, Rothman S M. The role of glutamate neurotoxicity in hypoxic-ischemic neuronal death. Annual review of neuroscience. 1990; 13:171-82). Moreover, ischemic-induced ATP reduction could lead to a collapse of the Na⁺/K⁺ electrochemical gradient and cause glutamate transporters to operate in the reverse direction (Rossi D J, Oshima T, Attwell D. Glutamate release in severe brain ischaemia is mainly by reversed uptake. Nature. 2000; 403(6767):316-21). A recent study pointed out that cystine-glutamate transporter (system x_{c}⁻)-mediated extrasynaptic glutamate release was a critical mechanism for elevating extracellular glutamate after oxygen and glucose deprivation (Soria F N, Perez-Samartin A, Martin A, Gona K B, Llop J, Szczupak B, et al. Extrasynaptic glutamate release through cystine/glutamate antiporter contributes to ischemic damage. The Journal of clinical investigation. 2014; 124(8):3645-55). These mechanisms contributed to a rapid and transient glutamate efflux and excitotoxicity during hypoxia or ischemia. However, the rise in extracellular glutamate levels was not a transient event and, in humans, was recorded for up to 4 days after acute ischemic stroke (Davalos A, Castillo J, Serena J, Noya M. Duration of glutamate release after acute ischemic stroke. Stroke; a journal of cerebral circulation. 1997; 28(4):708-10), suggesting other unknown mechanisms may be involved in the long-term elevation of extracellular glutamate and the resulting excitotoxicty.

Hypoxia-inducible factor 1 (HIF-1) is a key regulator in hypoxia and, due to the functions of its downstream genes, has been suggested to be an important mediator in neurological outcomes following stroke (Shi H. Hypoxia inducible factor 1 as a therapeutic target in ischemic stroke. Current medicinal chemistry. 2009; 16(34):4593-600). While the role of HIF-1 after stroke is debated, HIF-1α was up-regulated after cerebral ischemia and reperfusion (CIR) and mostly located in the penumbra, the salvageable tissue (Bergeron M, Yu A Y, Solway K E, Semenza G L, Sharp F R. Induction of hypoxia-inducible factor-1 (HIF-1) and its target genes following focal ischaemia in rat brain. The European journal of neuroscience. 1999; 11(12):4159-70). Interestingly, activation of HIF-1 could be rapidly increased within 1 h after CIR and lasted for up to 7-10 days (Bergeron M, Yu A Y, Solway K E, Semenza G L, Sharp F R. Induction of hypoxia-inducible factor-1 (HIF-1) and its target genes following focal ischaemia in rat brain. The European journal of neuroscience. 1999; 11(12):4159-70: Baranova O, Miranda L F, Pichiule P, Dragatsis I, Johnson R S, Chavez J C. Neuron-specific inactivation of the hypoxia inducible factor 1 alpha increases brain injury in a mouse model of transient focal cerebral ischemic. The Journal of neuroscience: the official journal of the Society for Neuroscience. 2007; 27(246320-32), suggesting this signaling plays a role in regulating the early and late events of brain injury and recovery after stroke. HIF-1 contributes to vasomotor control, angiogenesis, erythropoiesis, iron metabolism, cell proliferation/cell cycle control, cell death, and energy metabolism via regulation of a broad range of genes after CIR (Sharp F R, Bernaudin M. HIF1 and oxygen sensing in the brain. Nature reviews Neuroscience. 2004; 5(4437-48). However, it is still unclear whether HIF-1 plays a role in regulating glutamate homeostasis.

### SUMMARY

this disclosure, a method of treating oxygen glucose deprivation/re-oxygenation (OGDR)-induced cellular injury and apoptosis in neurons and astrocytes of a higher vertebrate animal is provided. The method comprises administering an effective amount of an inhibitor of cysteine-glutamate transporter (i.e. system x_{c}⁻) in the higher vertebrate animal to decrease a concentration of extracellular glutamate in the neurons and the astrocytes to treat the OGDR-induced cellular injury and apoptosis in the neurons and the astrocytes.

In one embodiment, the inhibitor comprises sorafenib or its derivative, regorafenib, which does not need to be administered with tissue-type plasminogen activator (abbreviated as tPA).

In another embodiment, the inhibitor comprises erastin.

In yet another embodiment, the inhibitor comprises sulfasalazine.

In yet another embodiment, the higher vertebrate animal is a mammal, such as a human.

In another aspect, a method of reducing cortical infarct volume in a brain of a higher vertebrate animal suffering ischemic or hypoxia brain injury is provided. The method comprises administering an effective amount of an inhibitor of cysteine-glutamate transporter (i.e. system x_{c}⁻) in the higher vertebrate animal, so that the cortical infarct volume in the brain is reduced.

In one embodiment, the inhibitor comprises sorafenib or its derivative, regorafenib, which does not need to be administered with tissue-type plasminogen activator (abbreviated as tPA).

In another embodiment, the inhibitor comprises erastin.

In yet another embodiment, the inhibitor comprises sulfasalazine.

In yet another embodiment, the higher vertebrate animal is a mammal, such as a human.

In yet another aspect, a method of reducing cerebral ischemia and reperfusion (CIR)-induced glutamate release as well as excitotoxicity to central nervous system (CNS) is provided. The method comprises administering an effective amount of an inhibitor of cysteine-glutamate transporter (i.e. system in the higher vertebrate animal to decrease a concentration of extracellular glutamate, so that the CIR-induced glutamate release as well as excitotoxicity to CNS is reduced.

In one embodiment, the inhibitor comprises sorafenib or its derivative, regorafenib, which does not need to be administered with tissue type plasminogen activator (abbreviated as tPA).

In another embodiment, the inhibitor comprises erastin.

In yet another embodiment, the inhibitor comprises sulfasalazine.

In yet another embodiment, the higher vertebrate animal is a mammal, such as a human.

In yet another aspect, a method of treating ischemic brain damage is provided. The method comprises administering an effective amount of an inhibitor of cysteine-glutamate transporter (i.e. system x_{c}⁻) in the higher vertebrate animal within 12 hours after the occurring of oxygen glucose deprivation.

In one embodiment, the inhibitor comprises sorafenib or its derivative, regorafenib, which does not need to be administered with tissue-type plasminogen activator (abbreviated as tPA).

In another embodiment, the inhibitor comprises erastin.

In yet another embodiment, the inhibitor comprises sulfasalazine.

In yet another embodiment, the higher vertebrate animal is a mammal, such as a human.

In the foregoing, the inhibitor of cysteine-glutamate transporter can improve or even treat ischemic brain damage, even though after the occurring of oxygen glucose deprivation for more than 3 hours, and even up to 12 hours. As for the conventional treatment method using tissue-type plasminogen activator (t-PA), t-PA needs to be administered with 3 hours after the occurring of oxygen glucose deprivation for effective treatment.

It is to be understood that both the foregoing general description and the following detailed description are by examples, and are intended to provide further explanation of the invention as claimed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 is a diagram of SLC1A1 SLC1A2, SLC1A3, and SLC7A11 mRNA levels in primary cortical cells exposed to oxygen glucose deprivation/re-oxygenation (OGDR) with or without YC-1 (5 μM);

FIG. 2 is a diagram of SLC1A1, SLC1A2, SLC1A3, and SLC7A11 mRNA levels in primary cortical cells at 18 h after transfection with control or HIF-1α-oxygen-dependent degradation domain deletion mutant (HIF-1α-ODDm) plasmids;

FIGS. 3A and 3B are diagrams of xCT mRNA and protein levels in homogenised ischemic brain tissue from rats after cerebral ischemia/reperfusion (CIR) treatment at the indicated times, respectively;

FIGS. 4A and 4B show immunofluorescence images of xCT expression in ischemic rat brains, and bars equal to 50 μm;

FIGS. 5A-5C demonstrate the overlay images of DAPI (blue), xCT (green), and neuronal nuclei (Neu-N, red, FIG. 5A), glial fibrillary acidic protein (GFAP, red, FIG. 5B) in or HIF-1α (red, FIG. 5C), and bars equal to 50 μm;

FIG. 6A shows photographs of postmortem brain slices from human ischemic stroke patients (n=4 patients) were stained for xCT immunoreactivity, and compared to control non-stroke patients died of glioblastoma multiforme (n=4 patients);

FIG. 6B is the quantitative result of FIG. 6A, and *P is <0.0001 compared to control, Student's t-test;

FIG. 7 shows the analytical results of tissue [¹⁴C] L-cystine radioactivity and extracellular glutamate levels in acute cortical slices from rats with CIR at the indicated time points after reperfusion;

FIGS. 8A and 8B show tissue [¹⁴C] L-cystine radioactivities (H) and extracellular glutamate levels (I) in acute cortical slices from rats with CIR 12 h after reperfusion in the presence of DMSO (vehicle); imatinib (10 μM), sorafenib (10 μM), regorafenib (10 μM), erastin (10 μM) or sulfasalazine (SAS) (500 μM) during Cl⁻-dependent [¹⁴C] L-cystine uptake and in vitro extracellular glutamate assays; respectively;

FIGS. 9A and 9B are diagrams showing xCT mRNA and protein levels in homogenised ischemic brain tissue derived from rats with or without pretreatment of 2-methoxyestradiol (2ME2, 150 mg/kg) followed by cerebral ischemia/reperfusion (CIR) 12 h after reperfusion;

FIGS. 10A and 10B show xCT mRNA and protein levels in neurons with or without HIF-1α or HIF-2α knockdown 24 h after OGDR, respectively. FIGS. 11A and 11B show xCT mRNA and protein levels in astrocytes with or without HIF-1α or HIF-2α knockdown 24 h after OGDR, respectively;

FIG. 12 shows graphic representation of the putative mouse and human xCT promoters;

FIG. 13 shows the results of the reporter activities of mouse xCT promoter in neurons and astrocytes with or without OGDR, desferrioxamine (DFO, 100 μM) or cobalt chloride (CoCl₂, 50 μM) incubation for 24 h;

FIG. 14 is a diagram showing the results of the promoter reporter assay of the neurons;

FIG. 15 is a diagram showing the results of the reporter activities of human xCT promoter in HEK-293 cells co-transfected with control (control v.) or HIF-1 subunits (HIF-1α or HIF-2α) plasmids and reporter plasmids for 48 h;

FIG. 16 shows the results of the HEK-293 cells co-transfected with the luciferase reporter plasmids carrying the wild type or HRE mutant human xCT promoter regions as well as the Renilla luciferase reporter plasmid, and then treated with or without OGDR for 24 h;

FIG. 17 is a diagram showing the results of ChIP followed by real-time PCR (ChIP-qPCR) assay of HIF-1α or HIF-2α binding in mouse xCT promoter in response to OGDR for 24 h;

FIG. 18A shows the results of intracellular glutathione level in wild type (WT) and xCT^{-/-} cortical cells treated with OGDR at 24 h after reperfusion;

FIG. 18B shows the results of extracellular glutamate content in wild type (WT) and xCT^{-/-} cortical′ cells treated with OGDR at 24 h after reperfusion;

FIG. 19 shows the results of binding radioactivity of ¹⁸F-FSAG in wild type (WT) and xCT^{-/-} cortical cells treated with OGDR at 24 h after reperfusion;

FIGS. 20A and 20B shows the results of LDH level and caspase-3 activity in wild type (WT) and xCT^{-/-} cortical cells treated with OGDR at 24 h after reperfusion;

FIGS. 21A and 21B show the results of apoptosis in wild type (WT) and xCT^{-/-} cortical cells treated with OGDR at 24 h after reperfusion;

FIGS. 22A to 22E are diagrams respectively showing the results of extracellular glutamate content, binding radioactivity of ¹⁸F-FSAG, lactate dehydrogenase (LDH) level, and apoptosis in WT cortical cells exposed to OGDR with or without vehicle, imatinib (10 μM), sorafenib (10 μM), regorafenib (10 μM), erastin (10 μM) or sulfasalazine (SAS, 500 μM) at 24 h after reperfusion;

FIG. 23 is a diagram show the results of lactate dehydrogenase (LDH) level in WT cortical cells treated with or without sorafenib (10 μM), erastin (10 μM) or sulfasalazine (SAS, 500 μM) at various time points after oxygen glucose deprivation (OGD).

FIG. 24 is a diagram showing the kinetics of extracellular glutamate content in ischemic cortex from wild type (WT) and xCT^{-/-} mice with cerebral ischemia/reperfusion (CIR);

FIG. 25 shows the accumulation of ¹⁸F-labelled alkylthiophenyl guanidine in the ipsilateral and contralateral cerebral hemispheres from WT and xCT^{-/-} mice with CIR at 12 h after reperfusion;

FIG. 26A shows ¹⁸F-FSAG PET imaging of brains in WT and xCT^{-/-} mice with CIR at 12 h after reperfusion;

FIG. 26B shows accumulation of ¹⁸F-FSAG in the ipsilateral and contralateral cerebral hemispheres of WT and xCT^{-/-} mice with CIR 12 h reperfusion;

FIGS. 27A and 27B respectively show representative photographs of 2,3,5-triphenyltetrazolium chloride (TTC) staining and calculated infarct volume in brains from WT and xCT^{-/-} mice with CIR 3 days post-reperfusion;

FIG. 28 shows extracellular glutamate content in ischemic cortex from rats with cerebral ischemia/reperfusion (CIR) followed by sulfasalazine (SAS) or the mixture of sulfapyridine (5-ASA) and salicylate (SP) as a control at 4 to 72 h after reperfusion;

FIG. 29 is a diagram showing suppression of glutamate efflux in ischemic cortex from rats with CIR followed by SAS at different dosages;

FIG. 30A is a diagram showing ¹⁸F-FSAG accumulation in the ipsilateral and contralateral cerebral hemispheres in the ipsilateral cerebral hemispheres of rats with CIR followed by SAS or the mixture of the mixture of 5-ASA and SP at 24 h after reperfusion;

FIG. 30B is a diagram showing caspase 3 activity in the ipsilateral cerebral hemispheres of rats with CIR followed by SAS or the mixture of the mixture of 5-ASA and SP at 24 h after reperfusion;

FIG. 30C is a diagram showing TUNEL-positive cells in the ipsilateral cerebral hemispheres of rats with CIR followed by SAS or the mixture of the mixture of 5-ASA and SP at 24 h after reperfusion;

FIG. 31 is a diagram showing inhibition of TUNEL-positive cells in the ipsilateral cerebral hemisphere of rats with CIR followed by SAS with different dosages;

FIGS. 32A and 32B respectively shows representative images of magnetic resonance images and calculated infarct volume in brains from rats with cerebral ischemia/reperfusion (CIR) followed by sulfasalazine (SAS) or the mixture of sulfapyridine (5-ASA) and salicylate (SP) on day 28 after reperfusion;

FIGS. 33A-33D are diagrams respectively showing body asymmetry, number of vertical movement, vertical activity, and vertical movement time in rats with CIR followed by SAS or the mixture of 5-ASA and SP on days 7, 14, 21, 28 after reperfusion;

FIG. 34 is a diagram showing grip strength ratio in rats with CIR followed by SAS or the mixture of 5-ASA and SP on day 28 after reperfusion;

FIG. 35 is a diagram showing representative photographs of TTC staining (g) in brains from rats with cerebral ischemia/reperfusion (CIR) followed by vehicle or sorafenib (30 mg/kg ip) for 3 days;

FIG. 36 is a diagram showing a working model of HIF-1-regulated system in CIR-mediated imbalance of glutamate homeostasis and excitotoxicity and its therapeutic innervation.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

### Materials and Methods

### Animals

All animals were treated according to the Institutional Guidelines of China Medical University and approved by the Institutional Animal Care and Use Committees of China Medical University. C57BL/6J mice used for wild-type were purchased from the Animal Facility of the National Science Counsel (NSC), xCT homozygous knockout (xCT^{-/-}) mice of 129/Svj-C57BL/6J mixed genetic background and their genotyping were as described previously (Sato H, Shiiya A, Kimata M, Maebara K, Tamba M, Sakakura Y, et al. Redox imbalance in cystine/glutamate transporter-deficient mice. The Journal of biological chemistry. 2005:280(45):37423-9, which is incorporated here by reference), xCT^{-/-} mice kindly provided by Dr. Hideyo Sato. These animals were used for isolation of primary cortical cells, neurons and astrocyte, brain slices, CIR models, microdialysis, biodistribution and positron emission tomography (PET) imaging studies. The adult male Sprague-Dawley rats (250-300 g, the Animal. Facility of the NSC) were also used for CIR models, magnetic resonance imaging (MRI) and behaviour studies.

### Primary Cortical Cells, Neurons and Astrocyte Preparation

Primary cortical cells were prepared from cerebral cortices of wild-type C57BL/6J or homozygote xCT^{-/-} mouse embryos in embryonic day 17 as previously described with modification (Goldberg M P, Choi D W. Combined oxygen and glucose deprivation in cortical cell culture: calcium-dependent and calcium-independent mechanisms of neuronal injury. The Journal of neuroscience: the official journal of the Society for Neuroscience, 1993; 13(8):3510-24, which is incorporated here by reference).

Dissected cortices were dissociated at 37° C. in Earl s balanced salt solution (EBSS) containing papain (50 U/ml) and DNase I (100 U/ml). Cells were replenished with MEM (invitrogen) containing 0.5 g/l BSA, 2% B27 supplement, 0.5 mM pyruvate and antibiotics. Finally, the culture medium was changed to serum free neurobasal medium containing 1 mM pyruvate, 1 mM glutamate, 0.5 g/l BSA, 2% B27 supplement, and antibiotics on the seventh day.

For neuronal cultures, cells were plated at a density of 2×10⁶ cells/cm² in poly-D-lysine coated plates (50 mg/ml) under serum-free conditions using neurobasal medium supplemented with B27, 2 mM glutamine, 25 μM glutamate and 25 mM β-mercapthoethanol. On the fourth day of plating, one-half of the medium was replaced with glutamate-free B27/neurobasal medium, and subsequently only glutamate-free medium was used to feed the cultures every 4 day. Experiments were performed in cells at day in vitro (DIV) 12.

For astrocyte cultures, cells were plated at a density of 1×10⁶ cells/cm² in 75 cm² flasks coated with poly-D-lysine (10 μg/ml) in minimal essential media supplemented with 10% fetal bovine serum, 5% horse serum, glutamine (2 mM), and sodium bicarbonate (25 mM). At confluence (DIV 7), glial cultures were shaken for 8 h at 200 rpm in a temperature-controlled incubator at 37° C. to dislodge cells that were loosely attached to the astrocyte monolayer. Cultures were maintained for an additional 3 days, detached with 0.05% trypsin/EDTA and used at DIV 15.

### Oxygen Glucose Deprivation/Re-Oxygenation (OGDR) Treatment

The cells cultured with glucose-free Earle's balanced salt solution (EBSS) were placed for 2 h within a hypoxic chamber (Bug Box; Ruskinn Technology) and continuously flushed with 95% N₂ and 5% CO₂ at 37° C. to maintain a pressure of gas-phase O₂ less than 1 mmHg (OM-14 oxygen monitor; SensorMedics Corporation).

Control cells were incubated in EBSS containing mM glucose in a normoxic incubator for the same time period.

### In Vivo Cerebral Ischema/Reperfusion (CIR)

Eight to ten-week-old male adult male Sprague-Dawley rats, C57BL/6J mice and xCT^{-/-} mice were anesthetized with 1.5% isoflurane in oxygen, allowed breathing spontaneously, and body temperature was maintained at 37° C. with a heat lamp during surgery for right middle cerebral artery ligation and bilateral common carotid artery clamping as previously described (Chen S T, Hsu C Y, Hogan E L, Maricq H, Balentine J D. A model of focal ischemic stroke in the rat: reproducible extensive cortical infarction. Stroke; a journal of cerebral circulation. 1986; 17(4):738-43, which is incorporated here by reference). Animals were subjected to transient cerebral ischemia for 90 min in rats and 2 h in mice.

### Real-Time Quantitative PCR (Q-PCR)

Q-PCR analysis was performed as described previously in Hsieh (Hsieh C H, Kuo J W, Lee Y J, Chang C W, Gelovani Liu R S. Construction of mutant TKGFP for real-time imaging of temporal dynamics of HIF-1 signal transduction activity mediated by hypoxia and reoxygenation in tumors in living mice. J Nucl Med. 2009; 50(12):2049-57, which is incorporated here by reference). Total RNA was isolated from cells or mice tissues. 1 μg DNse-treated RNA was converted to cDNA using SuperScript® III First-Strand Synthesis System (Invitrogen). The cDNA was then used for real time PCR quantification of mRNAs using the gene specific forward and reverse primers. The primers were:

Mouse SLC1A1
(SEQ ID NO: 1)
(F) 5′-ATTGGGCAGATCGTCACC-3′
and
(SEQ ID NO: 2)
(R) 5′-ACAGCACTCAGCACGATCAC-3′;
Mouse SLC1A2
(SEQ ID NO: 3)
(F) 5-GATGCCTTCCTGGATCTCATT-3′
and
(SEQ ID NO: 4)
(R) 5-TCTTTGTCACTGTCTGAATCTGC-3′;
Mouse SLC1A3
(SEQ ID NO: 5)
(F) 5′-CCGCTCGCTAAGCTGTTACT-3′
and
(SEQ ID NO: 6)
(R) 5′-CTTTGGTGTTAGAGAGGACAACTTT-3′;
Rat SLC7A11
(SEQ ID NO: 7)
(F) 5′-CAGAGCAGCCCTAAGGCACTTTCC-3′
and
(SEQ ID NO: 8)
(R) 5′-CCGATGACGGTGCCGATGATGATGG-3′;
Mouse SLC7A11
(SEQ ID NO: 9)
(F) 5′-CCTGGCATTTGGACGCTACAT-3′
and
(SEQ ID NO: 10)
(R) 5′-TGAGAATTGCTGTGAGCTTGCA-3′;
Rat 18S rRNA
(SEQ ID NO: 11)
(F) 5′-GCCCTATCAACTTTCGATGGTAGT-3′
and
(SEQ ID NO: 12)
(R) 5′-GGATGTGGTAGCCGTTTCTCA-3′;
and
Mouse 18S rRNA
(SEQ ID NO: 13)
(F) 5′-GTAACCCGTTGAACCCCATT-3′
and
(SEQ ID NO: 14)
(R) 5′-CCATCCAATCGGTAGTAGCG-3′.

### Western Blot Analysis

Western blot analysis was prepared as described previously (Hsieh C H, Kuo J W, Lee Y J, Chang C W, Gelovani J G, Liu R S. Construction of mutant TKGFP for real-time imaging of temporal dynamics of HIF-1 signal transduction activity mediated by hypoxia and reoxygenation in tumors in living mice. J Nucl Med. 2009; 50(12):2049-57, which is incorporated here by reference).

Cells were lysed in a homogenization buffer containing pepstatin (1.45 leupeptin (2.1 mM), dithiothreitol, triethanolamine (50 mM), and ethylenediamine tetraacetic acid/ethylene glycol tetraacetic acid (0.1 mM), Total protein (5-20 mg) was loaded in Laemmli buffer onto a 7.5% polyacrylamide stacking gel and run at 40 V and then 100 V through a 7.5% separating gel using a Mini Cell (Bio-Rad). The proteins were transferred to nitrocellulose membranes (Bio-Rad no. 162-0146) for 1 h using a Mini Trans-Blot Electrophoretic Transfer Cell (Bio-Rad) The membrane was then blocked in 13 PBS/0.05% polysorbate 20/5% nonfat dry milk overnight at 4° C. After 2 washes in 13 PBS/0.005% polysorbate 20, the monoclonal antibody was added for 1.5 h. After another 3 washes, secondary antibody was added for 1.5 h. Using an enhanced chemiluminescence kit (Amersham Life Science no. RPN2106), the membrane was developed on Kodak film in the dark room. The following antibodies were used: β-actin (Sigma-Aldrich, 1:10000 dilution) and xCT (Novus or GeneTex Inc., 1:1000 dilution).

### Immunofluorescence Imaging

Frozen brain sections were incubated with primary antibodies, xCT (1:250; Novus), HIF-1α (1:150; Novus), GFAP (1:400; Sigma-Aldrich) and Neu-N (1:200, Chemicon), overnight at 4° C. and secondary antibodies, Cy3, Cy5, or FITC-conjugated goat anti-rabbit or goat antibody (1:100; Molecular Probes). Tissue fluorescence was visualized with the Carl Zeiss LSM510 laser-scanning confocal microscope (ZEISS).

### Preparation of Brain Slices

Control and CIR-treated rats were anesthetized with CO₂ and rapidly decapitated. The brains were removed and transferred into an ice-cold artificial cerebral spinal fluid (ACSF). Brain tissues were cut transversely into slices of 300 μm and allowed to recover at 37° C. for 45 min in freshly ACSF. Slices were transferred to 24-well plates for Cl⁻-dependent [¹⁴C] L-cystine uptake and in vitro extracellular glutamate release assays.

Cl⁻-dependent [¹⁴C] L-cystine uptake

The activity of cystine/glutamate antiporter was performed using Cl⁻-dependent [¹⁴C] L-cystine uptake assay as described previously (Soria F N, Perez-Samartin A, Martin A, Dona K B, Llop J, Szczupak B, et al. Extrasynaptic glutamate release through cystine/glutamate antiporter contributes to ischemic damage. The Journal of clinical investigation. 2014; 124(8):3645-55, which is incorporated here by reference). Briefly, brain slices or primary cortical cells were incubated with 0.8 μM [¹⁴C] L-cystine (PerkinElmer) at 37° C. for 10 minutes. The uptake was terminated by rapidly rising cells two times with ice-cold unlabelled uptake buffer. The cells were then lysed by adding 0.8 ml of 0.2 N NaOH containing 1% SOS for radioactivity determination using a Tri-Garb B2910TR liquid scintillation analyzer (PerkinElmer). Briefly, brain slices or primary cortical cells were incubated with 0.8 μM[¹⁴C] L-cystine (PerkinElmer) at 37° C. for 10 min. The uptake was terminated by rapidly rising cells two times with ice-cold unlabelled uptake buffer. The cells were then lysed by adding 0.8 ml of 0.2 N NaOH containing 1% SOS for radioactivity determination using a Tri-Garb B2910TR liquid scintillation analyzer (PerkinElmer).

### Glutathione Detection Assay

ApoGSH™ Glutathione Detection Kit (BioVision) was used to evaluate cellular glutathione level according to the manufacturer's instruction.

### In Vitro Extracellular Glutamate Release Assay

Brain slices or primary cortical cells were incubated with 200 μl of buffer solution containing 5.33 mM KCl. 26.19 mM NaHCO, 117.24 mM NaCl, 1.01 mM NaH₂PO₄, 2.0 mM CaCl₂, 5.56 mM D-glucose, 100 μM cystine with or without 25 μM imatinib, 10 μM sorafenib, 10 μM regorafenib, 10 μM erastin or 500 μM sulfasalazine (SAS) incubated in 95% O₂ and 5% CO₂ for 1 h at 37° C.

### Glutamate Determination

Samples were diluted in 20 mM borate buffer at pH 9.0 and were derivatized for 1 min with N-tert-butyloxycarbonyl-L-cysteine and o-phthaldialdehyde. Samples then were separated in a 5-mm C18 reverse-phase column (220×4.6 mm) Sheri-5 (Brownlee), and glutamate was monitored by fluorescence (334 nm excitation and 433 nm emission) using an RF-10AXL fluorescence detector (Shimadzu). Standards of glutamate were assayed before and after the dialysis samples.

### Vector Constructions and Viral Transduction

The multiple cloning sites (MCS) of pTA-Luc vector (Clontech) was inserted with the cDNA fragment bearing −2000 to +1 bp mouse or human xCT promoter to drive the expression of firefly luciferase gene as pTA-mxCTp-Luc or pTA-hxCTp-Luc. The mutant of hypoxia response element on mouse or human xCT promoter was generated in the pTA-mxCTp-Luc or pTA-hxCTp-Luc as template by Quick Change Site-directed Mutagenesis Kit (Stratagene).

Full-length human HIF-1α or HIF-2α cDNA was amplified in a reaction with Platinum Taq DNA polymerase (Invitrogen) and was subcloned into pAS2.EYFP.puro (National RNAi core facility, Academia Sinica, Taiwan) at the NheI and EcoRI sites. Lentiviral vectors carrying short hairpin RNAs (shRNA)-targeting HIF-1α or HIF-2α and scrambled shRNA were provided by National RNAi core facility, Academia Sinica in Taiwan.

Lentivirus production and cell transduction were carried out according to protocols (Szulc J, Aebischer P. Conditional gene expression and knockdown using lentivirus vectors encoding shRNA. Methods in molecular biology. 2008; 434:291-309, which is incorporated here by reference). Briefly, human Embryonic Kidney 293T cells (HEK 293 cells) were plated and transfected with the (snRNA)-targeting HIF-1α or HIF-2α or scrambled shRNA and the virus packaging plasmid. Cells were plated and infected with lentiviruses expressing shRNA, in the presence of 8 ug/ml hexadimethrine bromide (polybrene) for 24 h, which was followed by puromycin (2 μg/ml; 48 h) selection. All constructs were confirmed by DNA sequencing.

### Promoter Reporter Assay

Cells were cotransfected with xCT promoter-driven reporter constructs with or without HRE mutation and Renilla reporter plasmids. At 24 h after transfection, the luciferase activity was examined by a dual luciferase reporter assay system (Promega) according to the manufacturers instructions, and firefly luciferase activity was normalized to the control renilla activity included in the kit. Luciferase activities are expressed as fold-increase over the luciferase activities in un-stimulated conditions.

### Chromatin Immunoprecipitation

Chromatin immunoprecipitation assays were performed using Imprint Chromatin Immunoprecipitation Kit (Sigma-Aldrich) according to the manufacturers protocol using an anti-HIF-1α or anti-HIF-2α antibody (Novus). PCR for the HRE in the mouse xCT promoter was performed with specific primers: (F) 5′-CTTATAGATCCAAAAAATAT-3 (SEQ ID NO: 15) and (R) 5′-AAATGAAGACCGAGTCCTTC-3′ (SEQ ID NO: 16), were used for the input DNA PCR product.

### Radiochemistry

Synthesis of ¹⁸F-labelled S-fluoroalkyl diarylguanidine-10 (¹⁸F-FSAG) was performed by ¹⁸F-fluorination of the protected precursor S-fluoroalkyl guanidine followed by acidic hydrolysis, as previously described (Robins E G, Zhao Y, Khan I, Wilson A, Luthra S K, Rstad E. Synthesis and in vitro evaluation of (18)F-labelled S-fluoroalkyl diarylguanidines; Novel high-affinity NMDA receptor antagonists for imaging with PET. Bioorganic & medicinal chemistry letters. 2010; 20(5):1749-51, which is incorporated here by reference). The radiochemical purity of ¹⁸F-FSAG was >95%.

### In Vitro ¹⁸F-FSAG Binding Assay

¹⁸F-FSAG (2 nM) was also treated into 96-well plates with the same dried. Then, 0.1 ml of 2N NaOH was added to each well to facilitate cell homogenization. The lysates were collected and counted using a γ-counter (Packard; Cobra).

### Apoptosis Assay

Annexin V staining was performed to determine cell apoptosis using the Annexin V-FITC Apoptosis Detection Kit (Sigma-Aldrich) for 10 min at room temperature according to the manufacturers instructions, and then flow cytometric analysis was performed.

### Microdialysis

A guide cannula guide (outer diameter: 0.65 mm) was implanted in ischemic cortex (2 mm caudal to the bregma, 2 mm lateral to the midline, and 1.5 mm ventral to the cortical surface) and secured to the skull with an anchor screw and acrylic dental cement. On the next day, a microdialysis probe (CMA10, Carnegie Medicin, Stockholm, Sweden; membrane length: 1 mm) was inserted and connected to a microinfusion pump set to a speed of 1 μl/min and then perfused with Ringer's solution (147 mM NaCl, 4 mM KCl, 2.3 mM CaCl₂). Samples were collected every 30 min for the duration of the experiment. Probe positioning was histologically verified at the end of the experiments.

### Biodistribution of ¹⁸F-FSAG

Animals received 29.6 MBq/kg of ¹⁸F-FSAG in 100 μl of PBS via lateral tail vein injection, and then were euthanized by CO₂/O₂ asphyxiation at 30 min after injection. After sacrifice, selected tissues of interest were then removed and weighed, and the radioactivity was measured using a γ-counter. The percentage injected dose per gram (% ID/g) was then calculated.

### MicroPET Imaging

Each subject was injected with 9.25 MBq of ¹⁸F-FSAG. At 30 min after injection, mice were scanned on a small-animal positron emission tomography (PET) scanner (microPET; Concorde Microsystems) under isoflurane anesthesia. Static images (30 min) were obtained with a zoom factor of 2 in a 256×256 matrix. Calculations were corrected for radiation decay of ¹⁸F and the amount of injected dose, and the consistent color scale was applied to all PET images.

### Measurement of Lactate Dehydrogenase (LDH) Activity

Lactate dehydrogenase activity were performed to determine cell apoptosis using the lactate dehydrogenase activity assay kit (BioVision) after the SAS and Sorafenib treatment (Shyu W C, Lin S Z, Chiang M E, Chen D C, Su C Y, Wang H J, et al. Secretoneurin promotes neuroprotection and neuronal plasticity via the Jak2/Stat3 pathway in murine models of stroke. The Journal of clinical investigation. 2008; 118(1):133-48, which is incorporated by reference).

### Triphenyltetrazolium Chloride (TTC) Staining

For Triphenyltetrazolium chloride staining, animals were perfused with saline. The brain tissue was removed, placed in cold saline for 5 minutes, and sliced into 2.0-mm-thick sections. The brain slices were incubated in 20 g/l triphenyltetrazolium chloride (Research Organics Inc.), dissolved in saline for 30 minutes at 37° C., and transferred to a 5% formaldehyde solution for fixation. The area of infarction in each slice was measured with a digital scanner (Shyu W C, Lin S Z, Chiang M F, Chen D C, Su C Y, Wang H J, et al. Secretoneurin promotes neuroprotection and neuronal plasticity via the Jak2/Stat3 pathway in murine models of stroke. The Journal of clinical investigation. 2008; 118(1):133-48, which is incorporated by reference).

### Caspase-3 Activity Assay

The caspase3 activity was performed on cells treated as described above using commercial kits (Bio-Rad) according to the manufacturer's instructions (Shyu W C, Lin S Z, Chiang M F, Chen D C, Su C Y, Wang H J, et al. Secretoneurin promotes neuroprotection and neuronal plasticity via the Jak2/Stat3 pathway in murine models of stroke. The Journal of clinical investigation. 2008; 118(1):133-48, which is incorporated by reference).

### TUNEL Histochemistry

To detect apoptosis, a TUNEL staining Kit (DeadEnd Fluorimetric TUNEL system; Promega) was used for the TUNEL assay (Shyu W C, Lin S Z, Chiang M F, Chen D C, Su C Y, Wang H J, et al. Secretoneurin promotes neuroprotection and neuronal plasticity via the Jak2/Stat3 pathway in murine models of stroke. The Journal of clinical investigation. 2008; 118(1):133-48, which is incorporated by reference).

After ischemia, rat brains were fixed by perfusion with saline and 4% paraformaldehyde. After brains had been frozen on dry ice, a series of adjacent 10-μm-thick sections were cut in the coronal plane with a cryostat. MRI was performed on rats under anesthesia in a General Electric imaging system (R4; GE) at 3.0 T. Brains were scanned in 6-8 coronal image slices, each 2 mm thick without any gaps. T2-weighted imaging pulse sequences were obtained with the use of a spin-echo technique (repetition time, 4,000 ms; echo time, 105 ms) and were captured sequentially for each animal at 1, 7, and 28 days after cerebral ischemia. To measure the infarction area in the right cortex, the noninfarcted area was subtracted in the right cortex from the total cortical area of the left hemisphere. The area of infarct was drawn manually from slice to slice, and the volume was then calculated by internal volume analysis software (Voxtool; GE) (Shyu W C, Lin S Z, Chiang M E, Chen D C, Su C Y, Wang H J, et al. Secretoneurin promotes neuroprotection and neuronal plasticity via the Jak2/Stat3 pathway in murine models of stroke. The Journal of clinical investigation. 2008; 118(1):133-48, which is incorporated by reference).

### Neurological Behavioral Measurements

Behavioral assessments were performed 3 days before cerebral ischemia and 72 hours after cerebral ischemia. The tests measured body asymmetry and locomotor activity. Furthermore, grip strength was analyzed using Grip Strength Meter (TSE-Systems) as previously described with modification. In brief, the percentage of improvement in grip strength was measured on each fore limb separately and was calculated as the ratio between mean strength of 20 pulls of the side contralateral to the ischemia and the ipsilateral side. In addition, the ratio of grip strength after treatment to baseline was also calculated, and changes were presented as percent of baseline (Shyu W C, Lin S Z, Chiang M F, Chen D C, Su C Y, Wang H J, et al. Secretoneurin promotes neuroprotection and neuronal plasticity via the Jak2/Stat3 pathway in murine models of stroke. The Journal of clinical investigation. 2068; 118(1):133-48, which is incorporated by reference).

### Animal Treatments

Rats were treated intraperitoneal injection with either vehicle, SAS (5 mg/kg/day) or the mixture of sulfapyridine (5-ASA, 3.12 mg/kg/day) and salicylate (SP, 1.72 mg/kg) for 3 days after brain ischemia. The daily dose will be divided into 2 doses (BID) with a 12 h-time interval to maintain the blood concentration of SAS according to previous pharmacokinetics studies (Chungi V S, Dittert L W, Shargel L. Pharmacokinetics of sulfasalazine metabolites in rats following concomitant oral administration of riboflavin. Pharmaceutical research. 1989; 6(12):1067-72 which is incorporated here by reference). For sorafenib treatment, rats were received intraperitoneal injection with vehicle or sorafenib (30 mg/kg) for 3 days after brain ischemia.

### Statistical Analysis

One-way analysis of variance with post hoc Scheffe analyses was carried out using the SPSS package (version 18.0). The differences between control and experimental groups were determined by the two-sided, unpaired Student t test. P<0.05 was considered significant.

### Results

Cerebral Ischemic/Reperfusion Promotes Long-Term xCT Expression and System x_{c}⁻ Function

SLC1A1 (EAAT3), SLC1A2 (GLT-1), SLC1A3 (GLAST-1), and SLC7A11 (xCT) regulate glutamate homeostasis through release and uptake of glutamate in neurons and astrocytes (Takahashi M, Billups B, Rossi D, Sarantis M, Hamann M, and Attwell D. The role of glutamate transporters in glutamate homeostasis in the brain. The Journal of experimental biology. 1997; 200(Pt 2):401-9; Schousboe A, and Waagepetersen H S. Role of astrocytes in glutamate homeostasis: implications for excitotoxicity. Neurotoxicity research. 2005; 8(3-4):221-5). To investigate which gene is the target gene of HIF-1, primary cortical cells were exposed to oxygen glucose deprivation/re-oxygenation (OGDR) with or without YC-1 (see chemical structure shown below), a small molecule inhibitor of HIF-1.

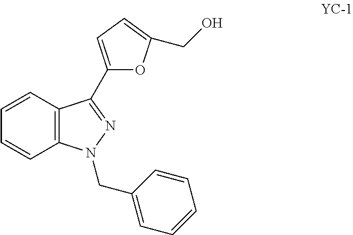 [SMILES: OCc1ccc(-c2nn(Cc3ccccc3)c3ccccc23)o1]

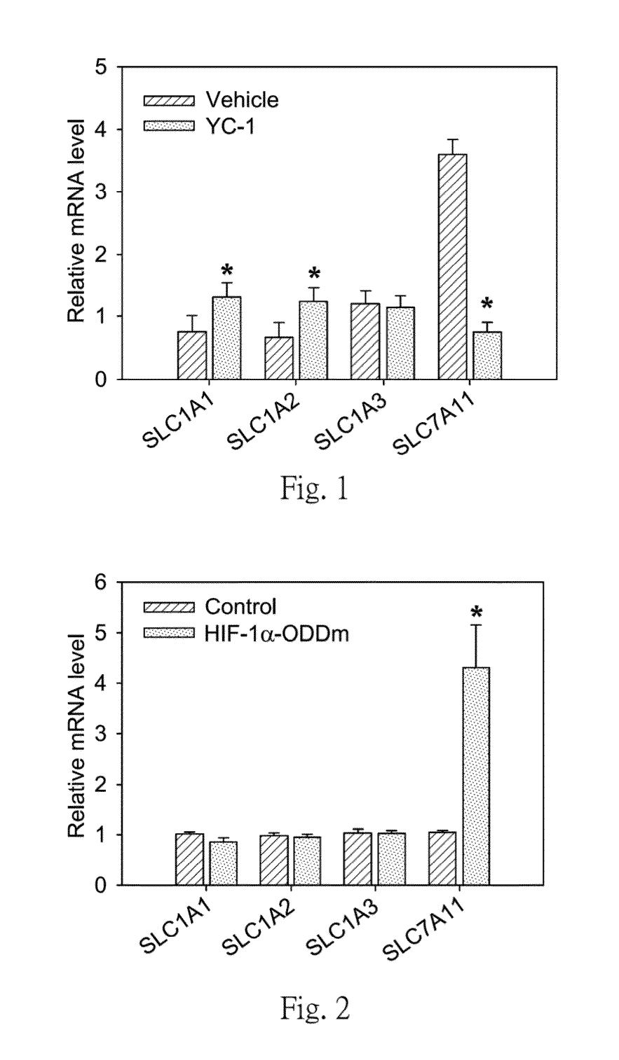

FIG. 1 is a diagram of SLC1A1 SLC1A2, SLC1A3, and SLC7A11 mRNA levels in primary cortical cells exposed to oxygen glucose deprivation/re-oxygenation (OGDR) with or without YC-1 (5 μM). In FIG. 1, mRNA levels are expressed relative to the corresponding mRNA level in the control condition without OGDR, and P is <0.001 compared to vehicle, Student's t-test. As evidenced by mRNA levels, OGDR suppressed SLC1A1 and SLC1A2 expression and increased SLC7A11 expression, and the latter was inhibited by pretreatment with YC-1.

FIG. 2 is a diagram of SLC1A1, SLC1A2, SLC1A3, and SLC7A11 mRNA levels in primary cortical cells at 18 h after transfection with control or HIF-1α-oxygen-dependent degradation domain deletion mutant (HIF-1α-ODDm) plasmids. In FIG. 2, *P is <0.0001 compared to control, Student's t-test. Transfection of plasmids carrying the HIF-1α-oxygen-dependent degradation domain deletion mutant significantly enhanced SLC7A11 expression, which did not occur in control plasmids. These results indicate that SLC7A11 is regulated by HIF-1.

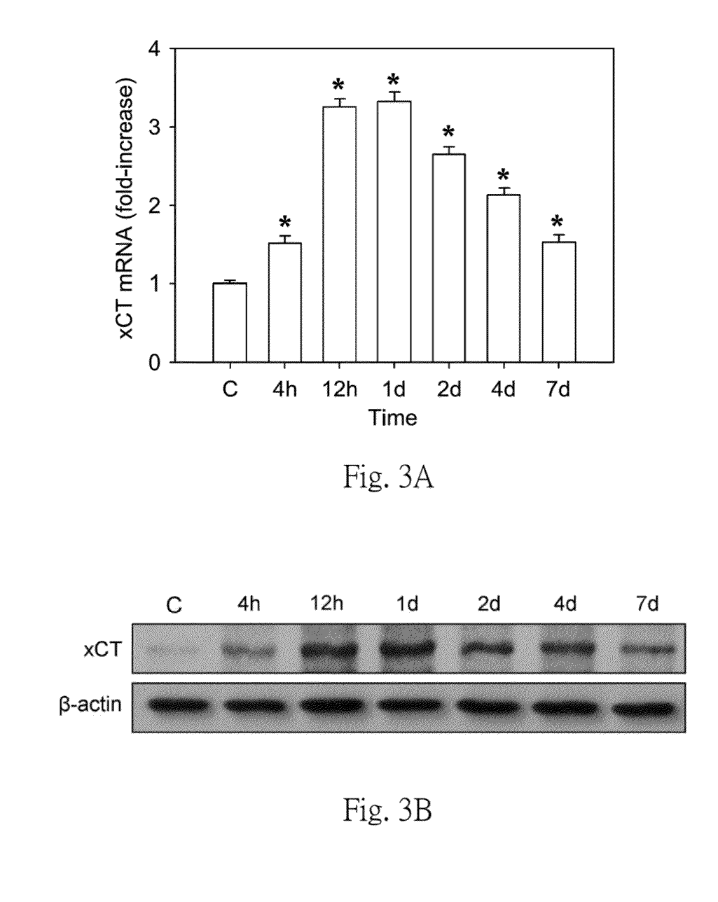

Next, a time course analysis of xCT expression during CIR in rats was performed. FIGS. 3A and 3B are diagrams of xCT mRNA and protein levels in homogenised ischemic brain tissue from rats after cerebral ischemia/reperfusion (CIR) treatment at the indicated times, respectively. Non-ischemic brain tissues were used as a control (C). *P is <0.001 compared to control, one-way ANOVA. In FIGS. 3A and 3B, xCT in ischemic brain tissues has a time-dependent increase in mRNA and protein levels with the peak of expression at 12-24 h after CIR and lasted for up to 7 days.

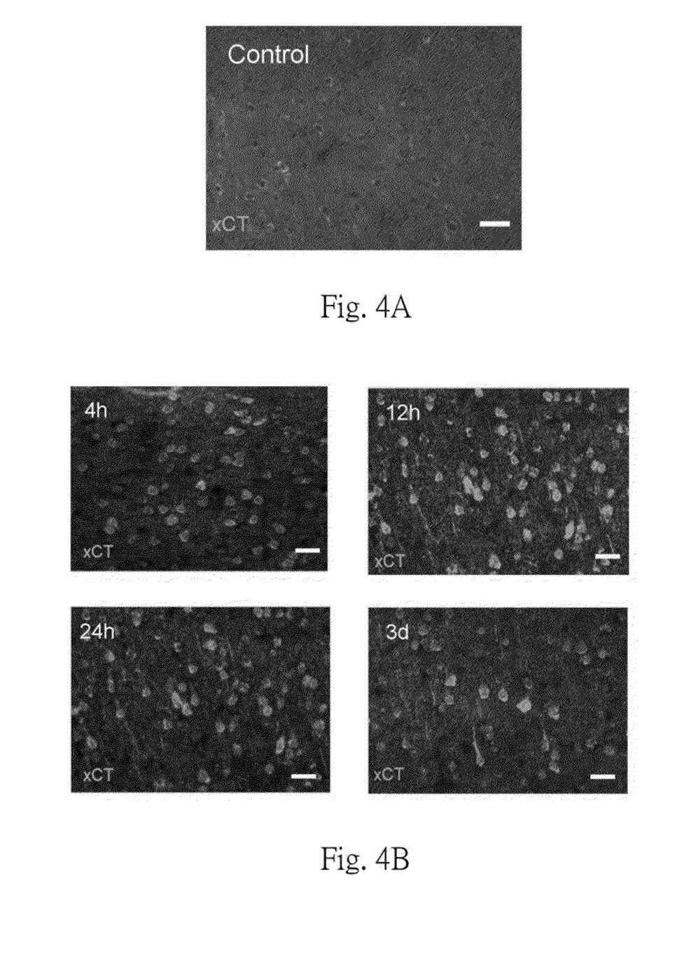

FIGS. 4A and 4B show immunofluorescence images of xCT expression in ischemic rat brains, and bars equal to 50 μm. FIG. 4A shows the staining result of a non-stroke rat brain. In FIG. 4A, it can be seen that the expression of xCT was scarcely seen in a non-stroke rat brain. FIG. 4B shows the staining of xCT (green) at the indicated time after the CIR treatment. In FIG. 4B, the localizations of the expressed xCT could be clearly seen in the ischemic rat brains, and the xCT expression in the ischemic rat brains reached maxima at 12-24 h after the CIR treatment.

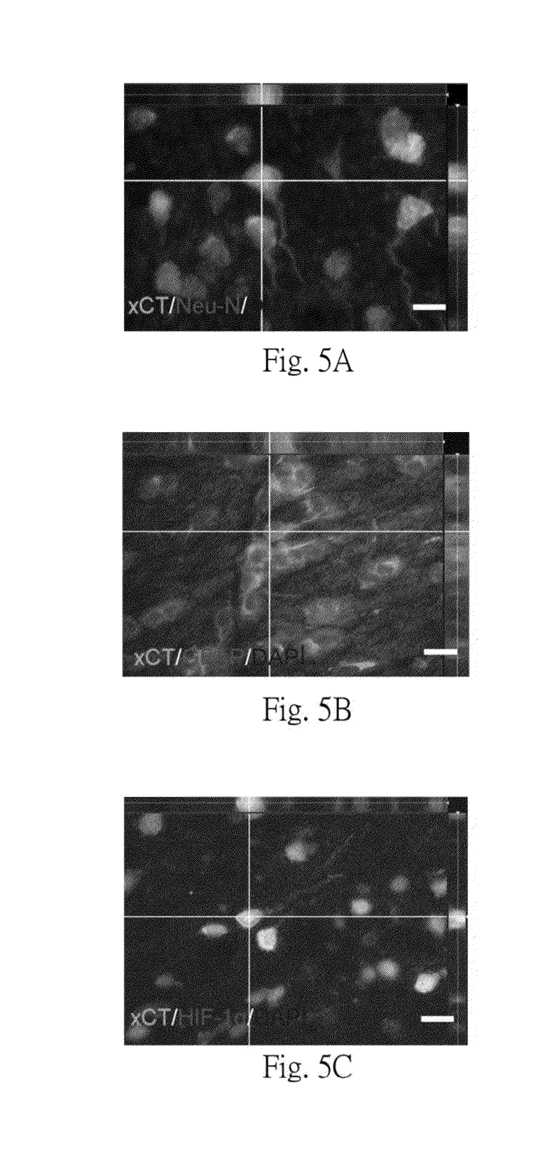

FIGS. 5A-5C demonstrate the overlay images of DAPI (blue), xCT (green), and neuronal nuclei (Neu-N, red, FIG. 5A), glial fibrillary acidic protein (GFAP, red, FIG. 5B) in or HIF-1α (red, FIG. 5C), and bars equal to 50 μm. The DAPI above is a fluorescent stain that binds strongly to A-T rich regions in DNA, and thus could be a nuclear marker. The immunofluorescence staining of xCT further revealed that xCT expression was colocalised with neuronal nuclei (Neu-N, a neuronal specific nuclear protein) and glial fibrillary acidic protein (GFAP, an astrocyte marker) in ischemic brain tissue, suggesting that both neurons and astrocytes increased xCT expression in response to CIR. Moreover, xCT expression was also colocalised with HIF-1α, indicating that CIR may regulate xCT expression via HIF-1 signalling.

To examine whether xCT expression is upregulated in stroke, postmortem brain tissues were collected from human patients that died from fatal ischemic stroke 1-3 day post ictus and nonischemic causes served as control as shown in our previous study (Lee S D, Lai T W, Lin S Z, Lin C H, Hsu V H, Li C Y, at al. Role of stress-inducible protein-1 in recruitment of bone marrow derived cells into the ischemic brains. EMBO molecular medicine. 2:013; 5(8):1227-46).

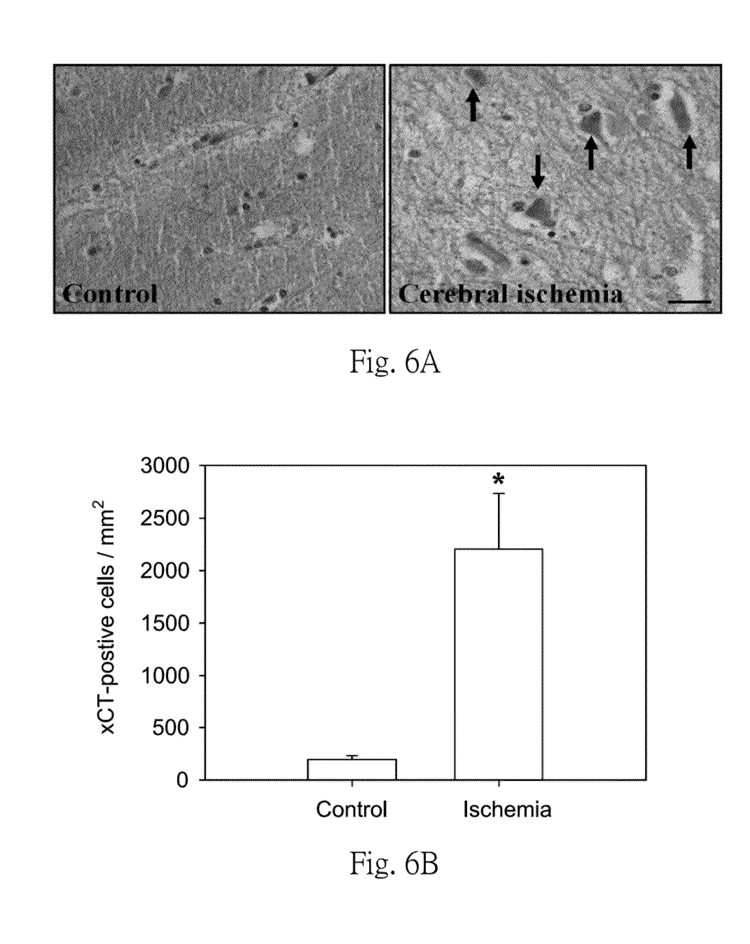

FIG. 6A shows photographs of postmortem brain slices from human ischemic stroke patients (n=4 patients) were stained for xCT immunoreactivity, and compared to control non-stroke patients died of glioblastoma multiforme (n=4 patients). Tissue sampling was based on individual infarct topography, and infarction was identified macroscopically. About 1 cm³ of cortical sample was dissected for analysis. In FIG. 6A, it can be seen that xCT-expressing cells (arrows pointing positions) were found in the penumbral region surrounding the ischemic infarct, which were extremely rare in the control brains.

FIG. 6B is the quantitative result of FIG. 6A, and *P is <0.0001 compared to control, Student's t-test. In FIG. 6B, significantly more xCT-positive cells were identified in the ischemic penumbra from stroke patients compared to similar area in control patients.

To determine whether CIR-induced xCT expression contributes to an alteration of system x_{c}⁻ function, the role of system x_{c}⁻ in cystine uptake and glutamate release after CIR were examined. Acute cortical slices from rats with CIR were prepared at several time points after reperfusion and subjected to Cl⁻-dependent [¹⁴C] L-cystine uptake and in vitro extracellular glutamate assays.

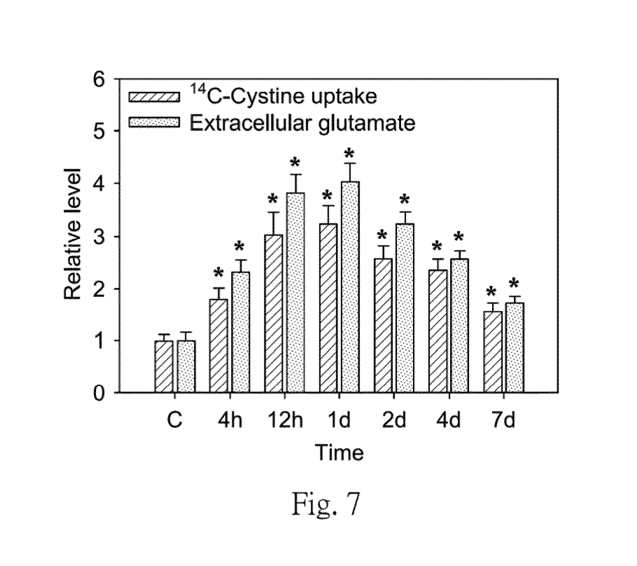

FIG. 7 shows the analytical results of tissue [¹⁴C] L-cystine radioactivity and extracellular glutamate levels in acute cortical slices from rats with CIR at the indicated time points after reperfusion. In FIG. 7, non-ischemic cortical slices were used as a control (C). From FIG. 7, it can be known that CIR-treated slices showed a time-dependent increase in tissue [¹⁴C] L-cystine and extracellular glutamate levels with the peak of expression at 12-24 h after CIR and continued for 7 days.

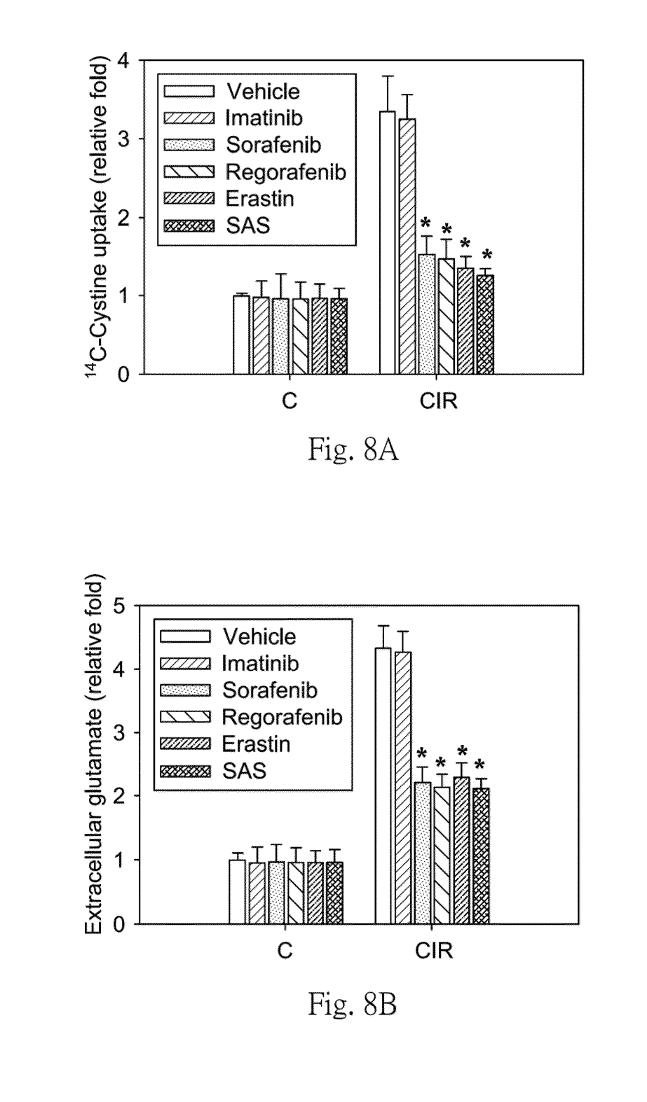

FIGS. 8A and 8B show tissue [¹⁴C] L-cystine radioactivities (H) and extracellular glutamate levels (I) in acute cortical slices from rats with CIR 12 h after reperfusion in the presence of DMSO (vehicle), imatinib (10 μM), sorafenib (10 μM), regorafenib (10 μM), erastin (10 μM) or sulfasalazine (SAS) (500 μM) during Cl⁻-dependent [¹⁴C] L-cystine uptake and in vitro extracellular glutamate assays, respectively. *P is <0.001 compared to control, one-way ANOVA. ^{#}P is <0.001 compared to CIR plus vehicle, one-way ANOVA. Error bars denote the standard deviation within triplicate experiments. The imatinib above is known as a tyrosine-kinase inhibitor but lacking system x_{c}⁻ inhibition activity (Dixon S J, Patel D N, Welsch M, Skouta R, Lee E D, Hayano M, et al. Pharmacological inhibition of cystine-glutamate exchange induces endoplasmic reticulum stress and ferroptosis. eLife. 2014; 3:e02523).

The chemical structure of imatinib, sorafenib, regorafenib, erastin, and sulfasalazine are shown below.

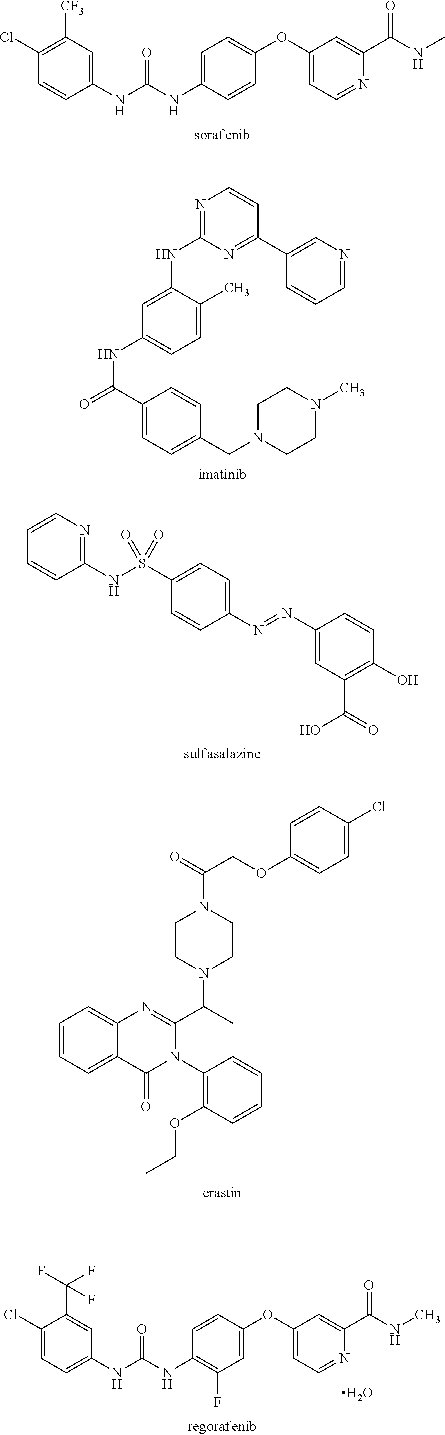

In FIGS. 8A and 8B, pharmacological blockade of system with selective inhibitors significantly attenuated CIR-induced elevation of tissue ¹⁴C-cystine radioactivity and extracellular glutamate content were compared to control vehicle or imatinib at 12 h after reperfusion. From the data of the control vehicle, the result indicates CIR promotes long-term xCT expression and system x_{c}⁻ function in neurons and astrocytes. Moreover, the tyrosine-kinase inhibitor lacking system x_{c}⁻ inhibition, imatinib, did not have any inhibiting effect on the uptake of cysteine and extracellular glutamate level. It can be seen that only sorafenib (10 μM), regorafenib (10 μM), erastin (10 μM) or sulfasalazine (SAS) (500 μM) could effectively decrease the cystine uptake and extracellular glutamate level.

### HIF-1α and HIF-2α Contribute to xCT Induction During OGDR

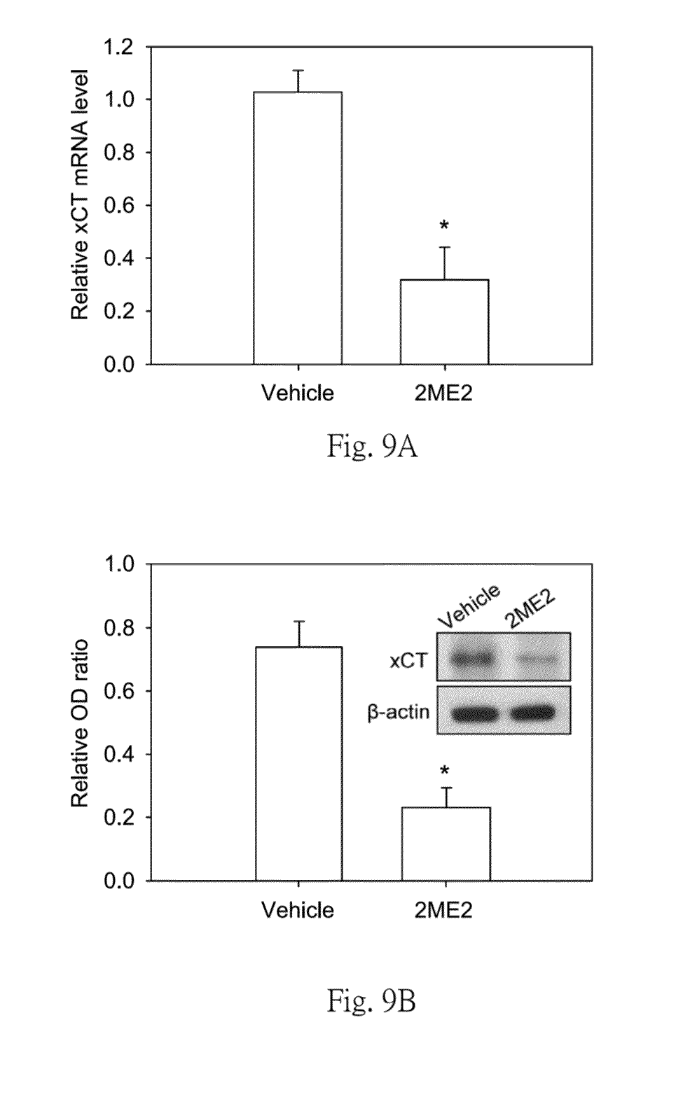

Because HIF-1 upregulated xCT expression, it was hypothesised that HIF members might participate directly in this process. To test this hypothesis, the mRNA and protein levels of xCT in ischemic brains with or without 2-methoxyestradiol (2ME2), an inhibitor of HIF-1, at 12 h after CIR were first determined. FIGS. 9A and 9B are diagrams showing xCT mRNA and protein levels in homogenised ischemic brain tissue derived from rats with or without pretreatment of 2-methoxyestradiol (2ME2, 150 mg/kg) followed by cerebral ischemia/reperfusion (CIR) 12 h after reperfusion. *P is <0.0001 compared to vehicle, Student's t-test. In FIGS. 9A and 9B, 2ME2 significantly inhibited xCT mRNA and protein expression levels, indicating that HIF-1 signalling plays an important role in CIR-mediated xCT induction.

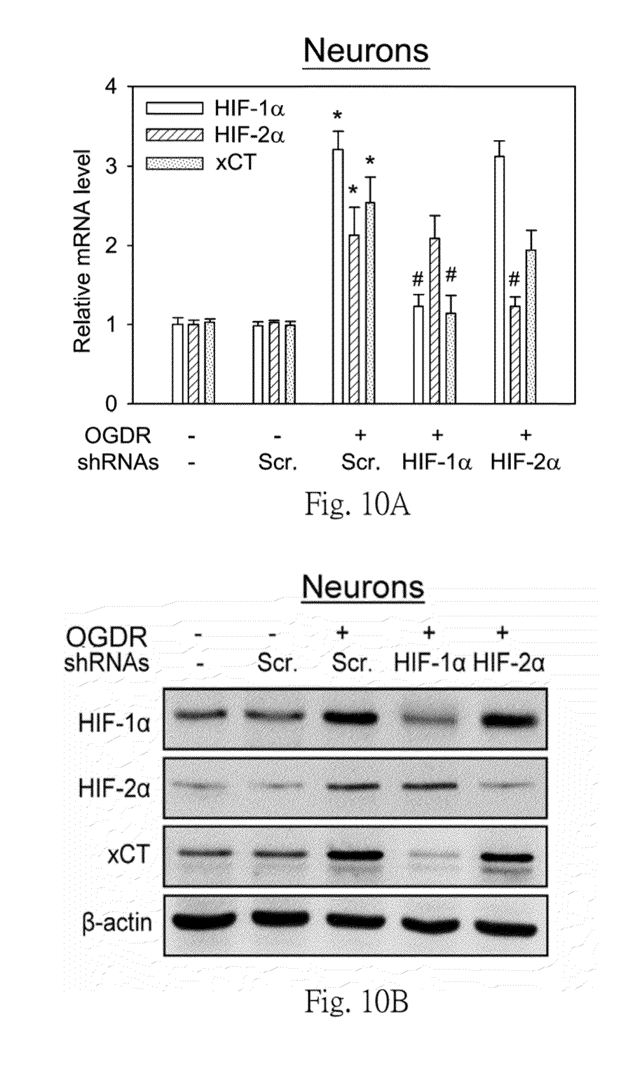

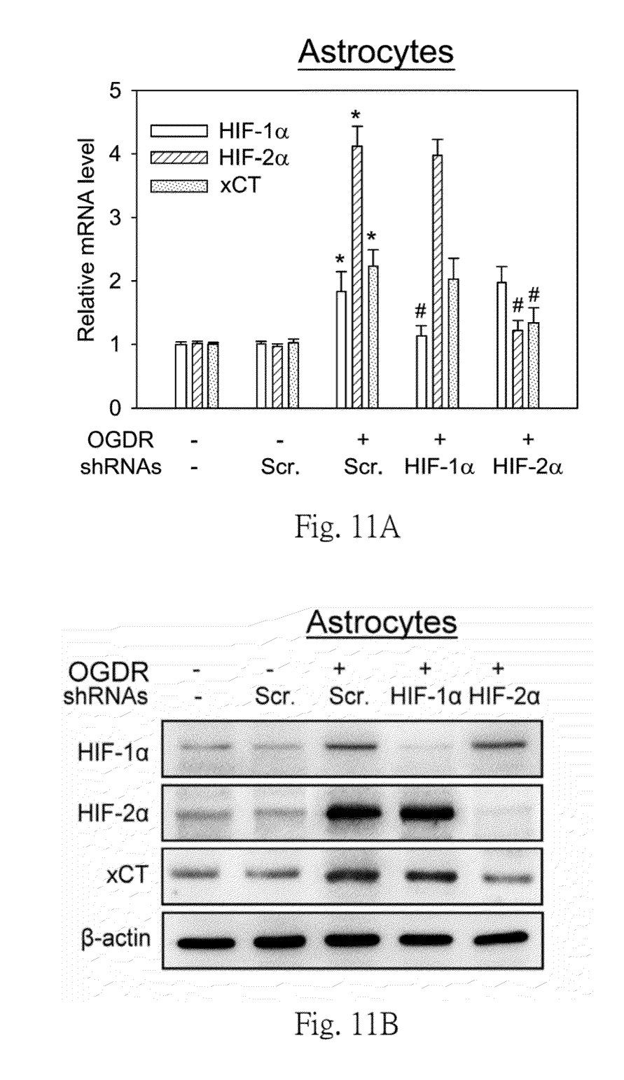

Next, primary cultures of neurons and astrocytes were exposed to OGDR. FIGS. 10A and 10B show xCT mRNA and protein levels in neurons with or without HIF-1α or HIF-2α knockdown 24 h after OGDR, respectively. FIGS. 11A and 11B show xCT mRNA and protein levels in astrocytes with or without HIF-1α or HIF-2α knockdown 24 h after OGDR, respectively. *P is <0.001 compared to control without OGDR, Student's t-test. P is <0.001 compared to OGDR with scramble (Scr.) shRNA, Student's t-test. In FIGS. 10A to 11B, the columns of “OGDR −/shRNAs−” show the results of samples not treated with both OGDR and shRNAs. The columns of “OGDR −/shRNAs Scr.” show results of samples not treated with OGDR but treated with scrambled shRNA. The columns of “OGDR +/shRNAs Scr.” show the results of samples treated with both OGDR and scrambled shRNAs. The columns of “OGDR +/shRNAs HIF-1α” show the results of samples treated with both OGDR and shRNA-targeting HIF-1α. Finally, the columns of “OGDR shRNAs HIF-2α” show the results of samples treated with both. OGDR and shRNA-targeting HIF-2α.

Comparing the results of columns “OGDR −/shRNAs −” and “OGDR +/shRNAs Scr.” in FIGS. 10A to 11B, the mRNA and protein levels were about the same. It shows that the scrambled shRNAs did not have any effects on the genes HIF-1α and HIF-2α. From the results of “OGDR shRNAs Scr,” it can be observed that mRNA and protein levels of xCT were upregulated by OGDR in neurons and astrocytes compared with controls. Moreover, protein levels of HIF-1α and, to a lesser degree, HIF-2α increased in response to OGDR in neurons. Conversely, in astrocytes exposed to OGDR, induction of HIF-2α was more prominent than HIF-1α induction.

Next, whether knockdown of endogenous HIF-α subunits affects endogenous xCT induction during OGDR was asked. The results are shown in columns “OGDR +/shRNAs HIF-1α” and “OGDR +/shRNAs HIF-2α”. From the results, it was observed that after lentiviral transduction with shRNAs against HIF-1α and HIF-2α, hypoxia-induced HIF-1α or HIF-2α expression was ablated in neurons and astrocytes compared with cells transduced with control scrambled shRNA. Knockdown of HIF-1α, but not HIF-2α, significantly abrogated OGDR-induced xCT expression in neurons whereas knockdown of HIF-2α, but not HIF-1α, predominantly inhibited OGDR-induced xCT expression in astrocytes, suggesting neurons and astrocytes rely preferentially on different HIF-1α subunits to drive OGDR-dependent xCT expression.

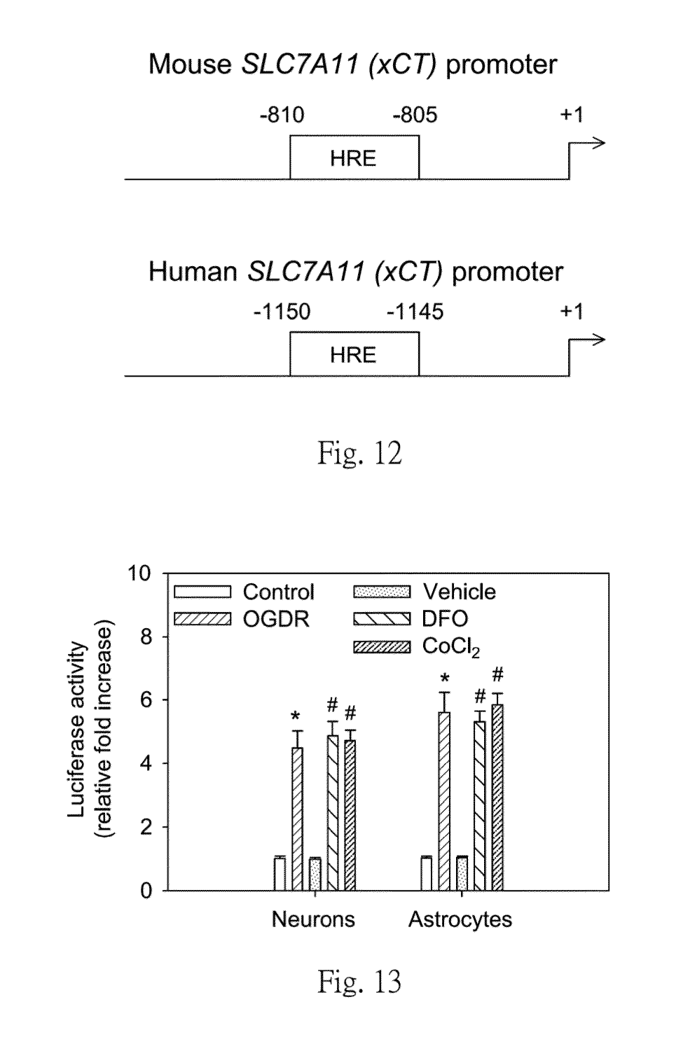

Next, whether HIF-1α or HIF-2α binds to the xCT promoter for OGDR-induced expression was determined. A bioinformatics analysis identified one hypoxia response element (HRE) in the mouse and human xCT promoter sequences from −2000 to +1 base pairs (bp), suggesting that HIF-1α subunits might regulate xCT expression by directly binding to the xCT promoter. The graphic representation of the putative mouse and human xCT promoters is shown in FIG. 12.

To test whether the xCT promoter would respond to HIF activation, the mouse xCT 2000-bp promoter was isolated and fused to firefly luciferase coding sequences for use in transient transfection assays with neurons and astrocytes. Normoxic neurons and astrocytes were treated with OGDR or incubated with desferrioxamine (DFO; 100 μM) or cobalt chloride (CoCl₂; 50 μM) for 24 h. The DFO and CoCl₂ mimics hypoxia by inducing transcription from HIF-1-dependent genes.

FIG. 13 shows the results of the reporter activities of mouse xCT promoter in neurons and astrocytes with or without OGDR, desferrioxamine (DFO, 100 μM) or cobalt chloride (CoCl₂, 50 μM) incubation for 24 h. *P is <0.0001 compared to control without OGDR Student's t-test. ^{#}P is <0.0001 compared to vehicle, Student's t-test. Comparing with the control and Vehicle (DMSO) groups, the groups of DFO and CoCl₂ increased the transcriptional activation of xCT to a level similar to that found during OGDR.

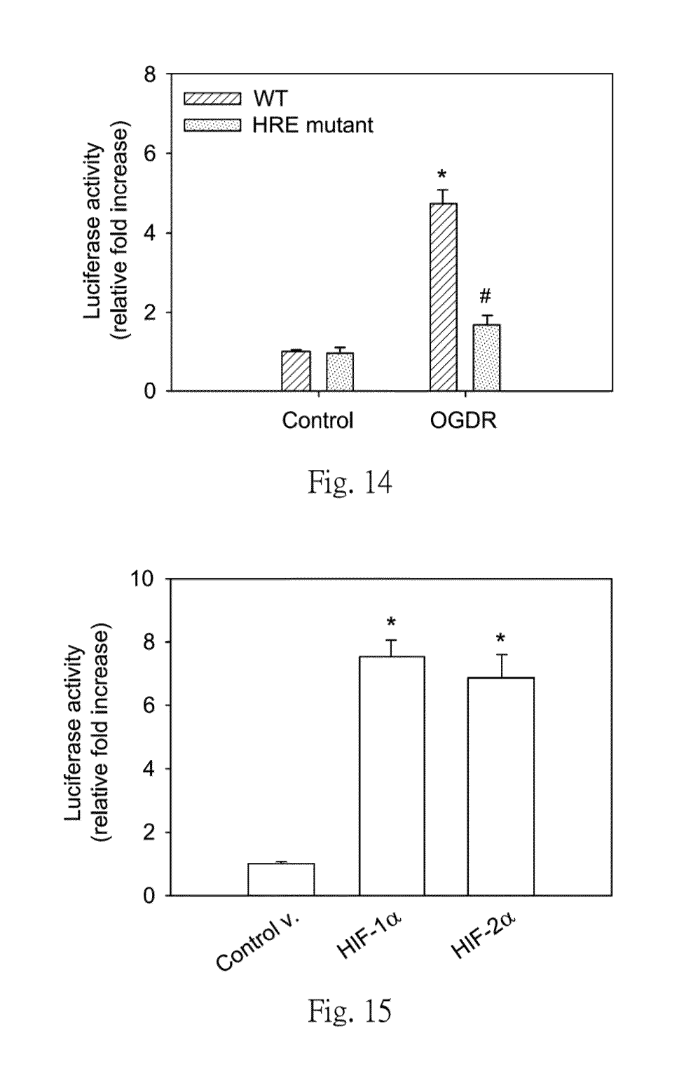

To pinpoint the exact binding motif, a point mutation was introduced into the HRE of the mouse xCT promoter. Then, luciferase reporter plasmids carrying the wild type or HRE mutant mouse xCT promoter regions were co-transfected with the Renilla luciferase reporter plasmid into mouse neurons. Next, the neurons were treated with or without OGDR for 24 h. FIG. 14 is a diagram showing the results of the promoter reporter assay of the neurons. *P is <0.0001 compared to control without OGDR, Student's t-test. ^{#}P is <0.0001 compared to wild type with OGDR, Student's t-test. In FIG. 14, it is observed that the HRE mutation of the mouse xCT promoter abolished the OGDR-mediated xCT induction in neurons. A similar effect was observed in the human xCT promoter.

Next, coexpression of HIF-1α or HIF-2α and the human xCT promoter was performed. FIG. 15 is a diagram showing the results of the reporter activities of human xCT promoter in HEK-293 cells co-transfected with control (control v.) or HIF-1 subunits (HIF-1α or HIF-2α) plasmids and reporter plasmids for 48 h. *P<0.0001 compared to control plasmids, Student's t-test. It can be observed that the coexpression of HIF-1α or HIF-2α and the human xCT promoter-driven luciferase reporter significantly enhanced the reporter activity but not the control plasmids in HEK293 cells.

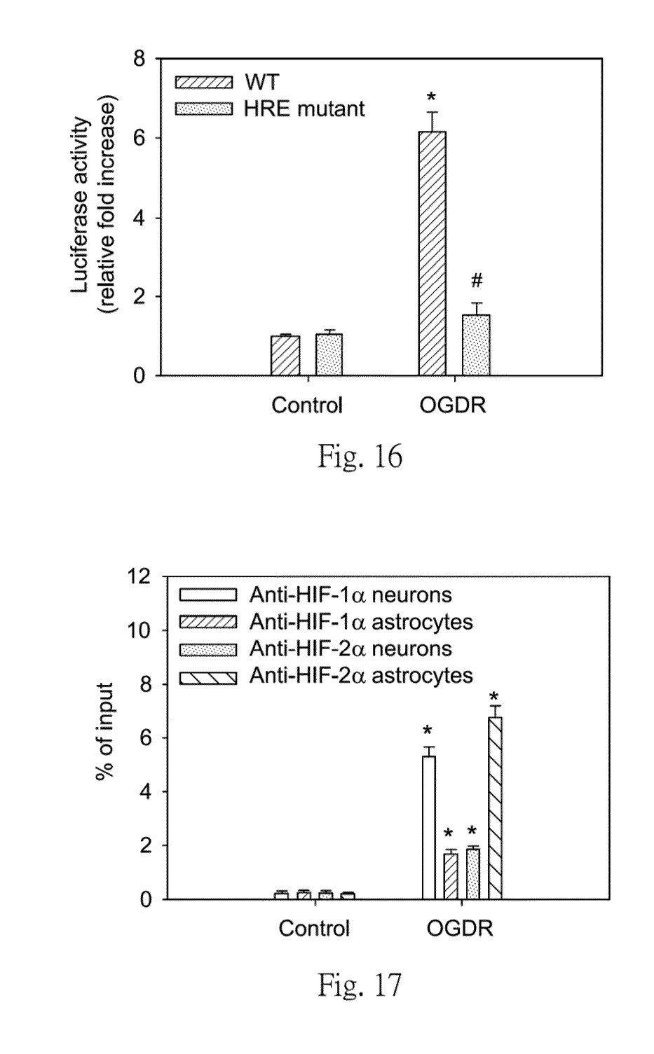

Furthermore, luciferase reporter plasmids carrying the wild type or HRE mutant human xCT promoter regions were co-transfected with the Renilla luciferase reporter plasmid into HEK-293 cells; and the cells were treated with or without OGDR for 24 h. FIG. 16 shows the results of the HEK-293 cells co-transfected with the luciferase reporter plasmids carrying the wild type or HRE mutant human xCT promoter regions as well as the Renilla luciferase reporter plasmid, and then treated with or without OGDR for 24 h. *P<0.0001 compared to control without OGDR, Student's t-test. ^{#}P<0.0001 compared to wild type with OGDR, Student's t-test. In FIG. 16, it was observed that the HRE mutation in the human xCT promoter inhibited its promoter activity and thus the luciferase activity.

Chromatin immunoprecipitation (ChIP) assays were also performed to investigate the interaction between HIF-1α and HIF-2α with mouse xCT promoter n neurons and astrocytes. FIG. 17 is a diagram showing the results of ChIP followed by real-time PCR (ChIP-qPCR) assay of HIF-1α or HIF-2α binding in mouse xCT promoter in response to OGDR for 24 h. Results are expressed as percentage of input. *P is <0.001 compared to control without OGDR, Student's t-test. Error bars denote the standard deviation among triplicate experiments. The results in FIG. 17 also confirmed the binding of HIF-1α and HIF-2α to mouse xCT promoter in neurons and astrocytes.

Collectively, these results suggest that HIF-1α and HIF-2α regulate xCT transcription by directly binding to the xCT promoter in an OGDR-dependent fashion.

Genetic Deficiency and Pharmacological Inhibition of System x_{c}⁻ Protects Primary Cortical Cells During OGDR

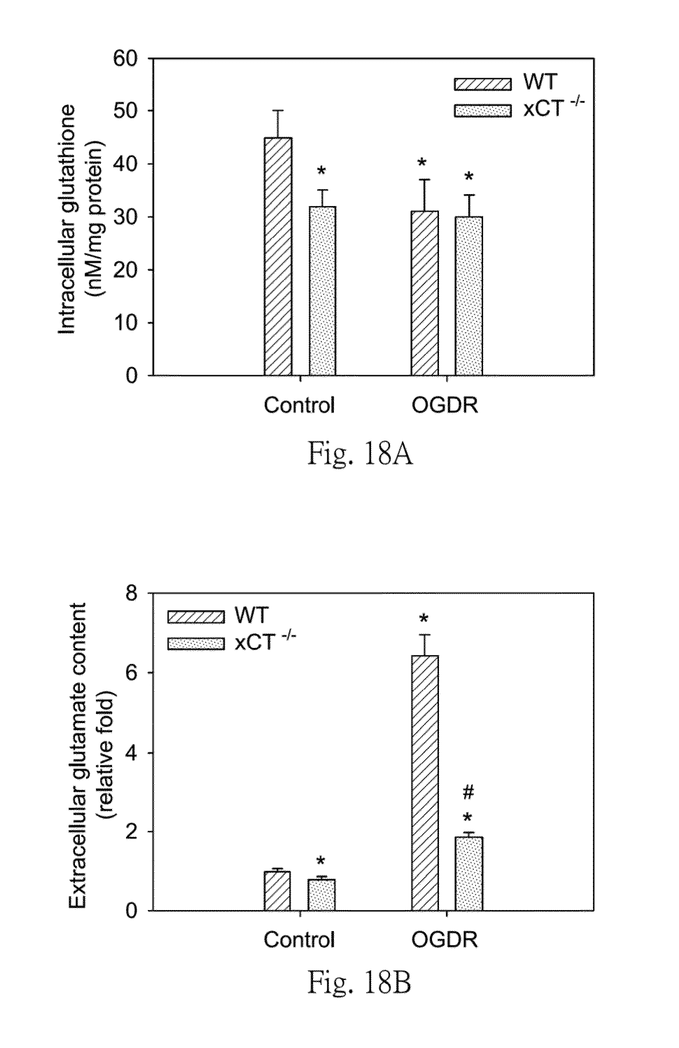

Because of the influence of activated system x_{G} on intracellular glutathione synthesis and nonvesicular glutamate release, the effects of system x_{c}⁺ deficiency after OGDR on intracellular glutathione and extracellular glutamate levels in primary cortical cells from xCT^{-/-} mice was examined. FIG. 18A shows the results of intracellular glutathione level in wild type (WT) and xCT^{-/-} cortical cells treated with OGDR at 24 h after reperfusion. *P<0.01 compared to WT without OGDR, Student's t-test. In FIG. 18A, although the endogenous glutathione in wild type cortical cells was higher than in xCT^{-/-} cortical cells, OGDR in wild type cortical cells decreased the intracellular glutathione level, but this effect was not observed in xCT^{-/-} cortical cells

FIG. 18B shows the results of extracellular glutamate content in wild type (WT) and xCT^{-/-} cortical cells treated with OGDR at 24 h after reperfusion. *P<0.01 compared to WT without OGDR, Student's t-test. ^{#}P<0.01 compared to WT with OGDR. Student's t-test. There was no significant difference in intracellular glutathione levels between wild type and xCT^{-/-} cortical cells exposed to OGDR, but OGDR largely increased extracellular glutamate content in wild type but not in xCT^{-/-} cortical cells. It suggests system x_{c}⁻ plays an important role in OGDR-induced glutamate release. The increased extracellular glutamate levels may lead to excitotoxicity via N-methyl-D-aspartate receptor (NMDAR) activation. Therefore, the possible contribution of system x_{c}⁻ to hyperfunction of NMDAR during OGDR was analysed.

A radiotracer, ¹⁸F-labelled alkylthiophenyl guanidine (¹⁸F-FSAG), which a specific radioligand for PCP sites of the NMDA receptor and thus binds to the PCP site of the NMDA channel (Robins E G, Zhao Y, Khan I, Wilson A, Luthra S K, Rstad E. Synthesis and in vitro evaluation of (18)F-labelled S-fluoroalkyl diarylguanidines: Novel high-affinity NMDA receptor antagonists for imaging with PET. Bioorganic & medicinal chemistry letters. 2010; 20(5):1749-51), was synthesised for observing the activation of NMDAR in vitro and in vivo.

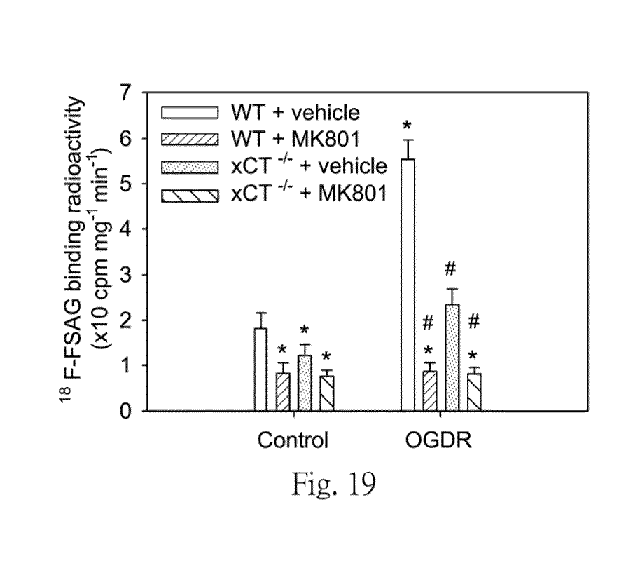

FIG. 19 shows the results of binding radioactivity of ¹⁸F-FSAG in wild type (WT) and xCT^{-/-} cortical cells treated with OGDR at 24 h after reperfusion. *P<0.01 compared to WT without OGDR, Student's t-test. ^{#}P<0.01 compared to WT with OGDR, Student's t-test. In FIG. 19, OGDR significantly increased the binding radioactivity of ¹⁸F-FSAG in wild type cortical cells, while genetic deficiency of xCT in cortical cells largely inhibited this effect. Moreover, pretreatment of cortical cells with MK801 (10 μM), a non-competitive antagonist of the NMDAR, blocked the binding of ¹⁸F-FSAG, indicating radioligand specificity for the activation of NMDAR. These results suggest system x_{c}⁻ plays a critical role in OGDR-induced hyperfunction of the NMDAR.

Next, we tested whether genetic deficiency of xCT protects cortical cells exposed to OGDR. Lactate dehydrogenase (LDH, a marker for cell apoptosis), caspase-3 activity (an important enzyme in the cell apoptosis), and apoptosis assays were used to observe cellular injury and apoptosis in cortical cells with or without genetic deficiency of xCT after OGDR.

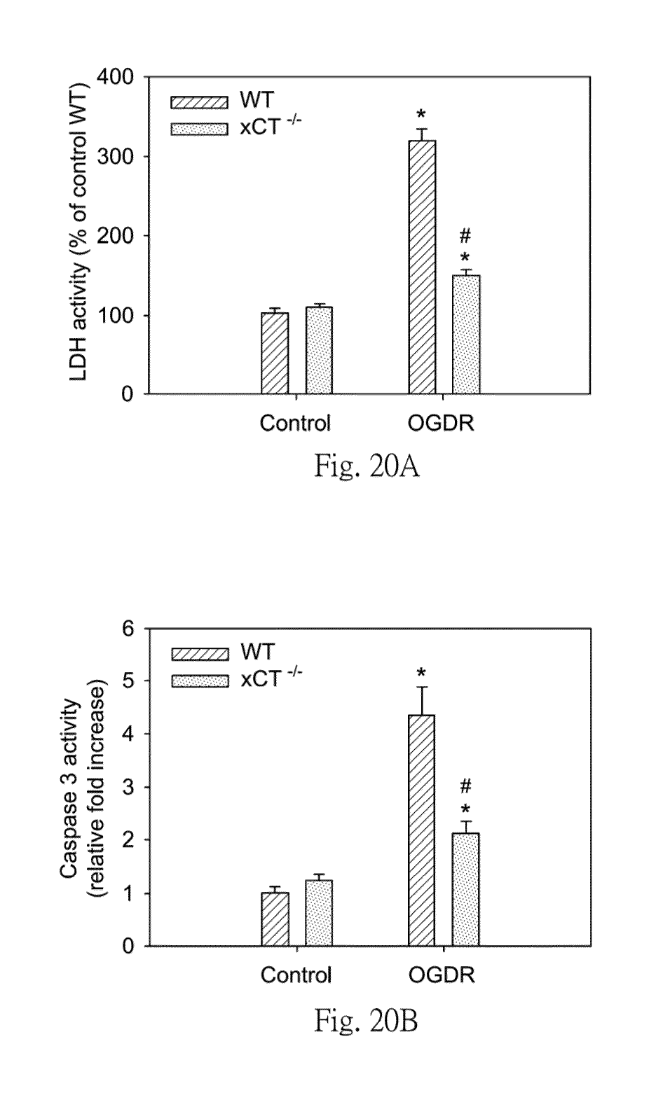

FIGS. 20A and 20B shows the results of LDH level and caspase-3 activity in wild type (WT) and xCT^{-/-} cortical cells treated with OGDR at 24 h after reperfusion. *P<0.01 compared to WT without OGDR Student's t-test. ^{#}P<0.01 compared to WT with OGDR, Student's t-test. In FIGS. 20A and 206, it was observed that xCT^{-/-} cortical cells had lower LDH levels and caspase-3 activity after OGDR compared to wild type cortical cell.

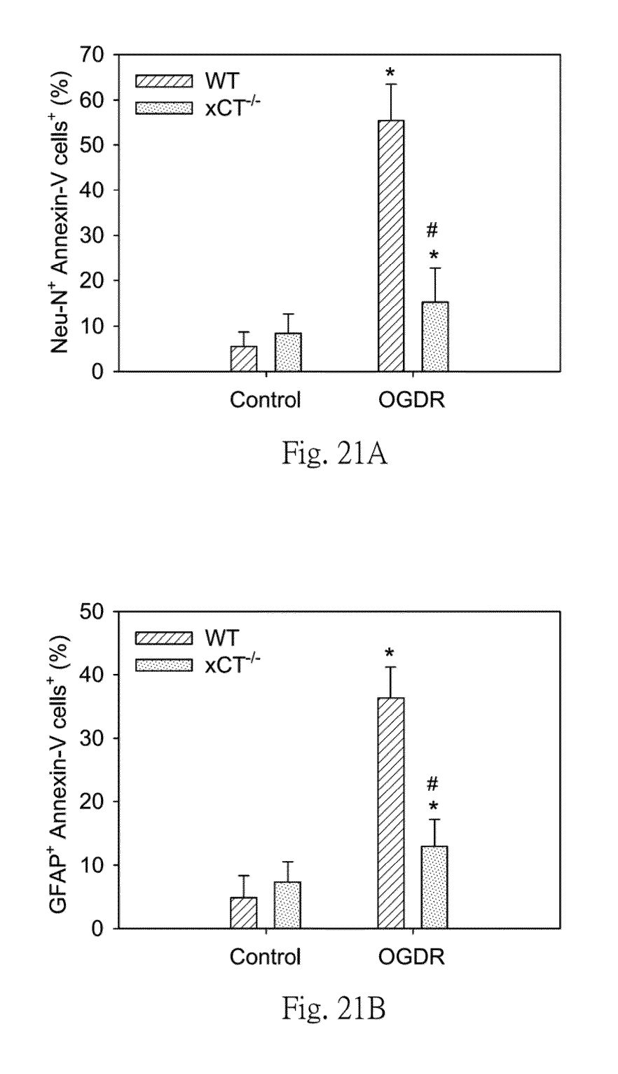

The three-color staining flow cytometric assay using allophycocyanin-microtubule-associated protein 2 (MAP2) for neuron staining, phycoerythrin (PE)-GFAP for astrocyte staining and FITC-annexin V for apoptotic cell staining was performed to count the numbers of neurons, astrocytes, and apoptotic cells. FIGS. 21A and 216 show the results of apoptosis in wild type (WT) and xCT^{-/-} cortical cells treated with OGDR at 24 h after reperfusion. *P<0.01 compared to WT without OGDR, Student's t-test. ^{#}P<0.01 compared to WT with OGDR, Student's t-test. From FIGS. 21A and 21B it was observed that that smaller numbers of apoptotic cells were present in xCT^{-/-} neurons and astrocytes after OGDR treatment. This result confirmed that the absence of system x_{c}⁻ prevented neuronal and astrocytic death in cortical cells after OGDR.

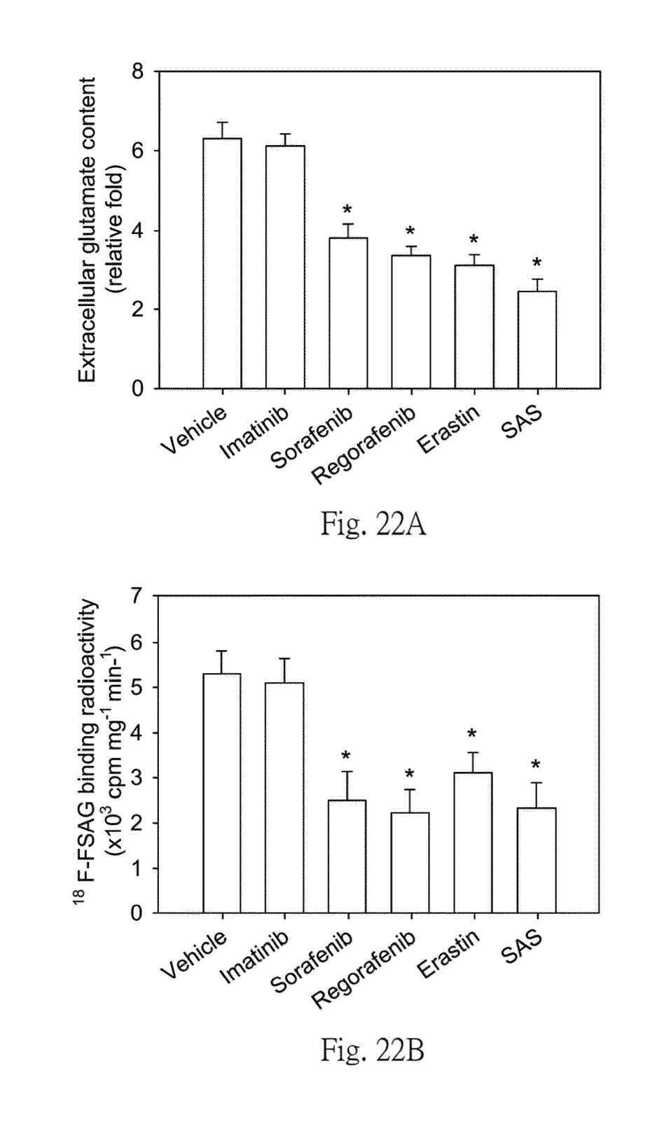

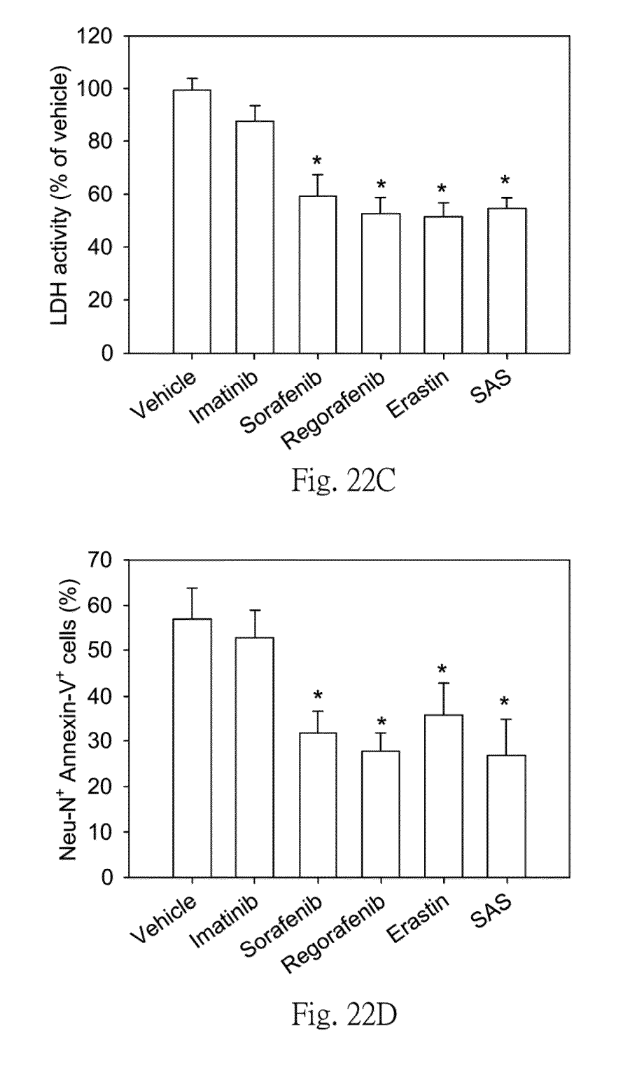

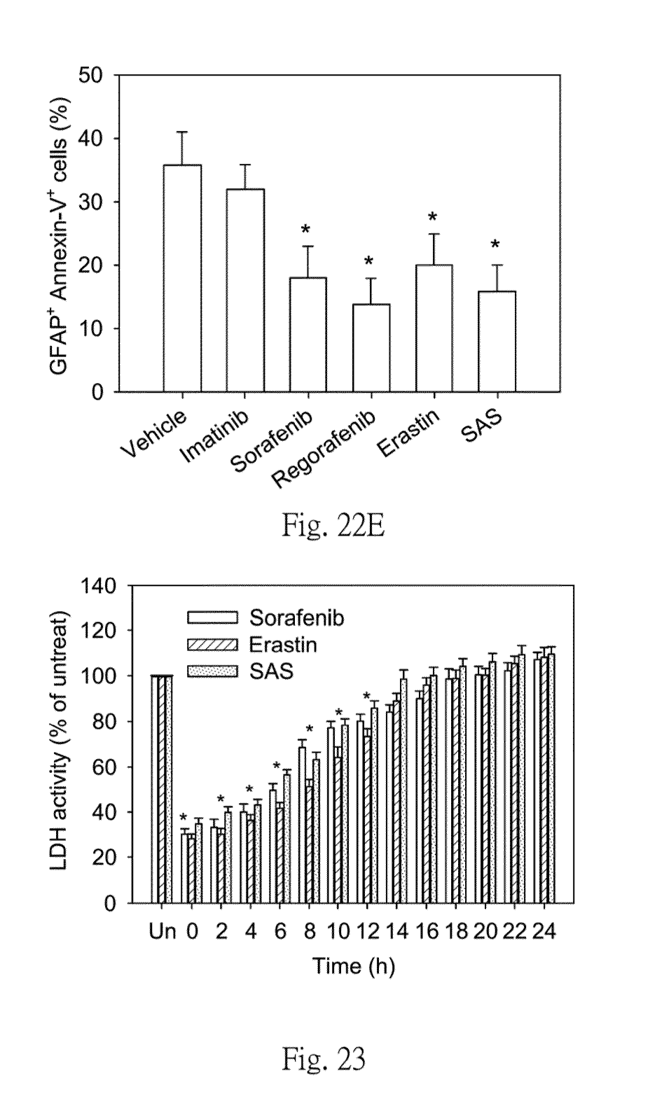

To test whether pharmacological inhibition of system x_{c}⁻ also had similar biological effects, wild type cortical cells were treated with known inhibitors of system x_{c}⁻ (i.e. sorafenib, erastin and SAS; please see Dixon S J, Patel D N, Welsch M, Skouta R, Lee E D, Hayano M, et al. Pharmacological inhibition of cystine-glutamate exchange induces endoplasmic reticulum stress and ferroptosis. eLife. 2014; 3:e02523) during OGDR. The results are shown in FIGS. 22A to 22E.

FIGS. 22A to 22E are diagrams respectively showing the results of extracellular glutamate content, binding radioactivity of ¹⁸F-FSAG, lactate dehydrogenase (LDH) level, and apoptosis in WT cortical cells exposed to OGDR with or without vehicle, imatinib (10 μM), sorafenib (10 μM), regorafenib (10 μM), erastin (10 μM) or sulfasalazine (SAS, 500 μM) at 24 h after reperfusion. *P is <0.001 compared to vehicle, one-way ANOVA. Error bars denote the standard deviation among triplicate experiments. It was observed that sorafenib, regorafenib, erastin or SAS treatment inhibited OGDR-induced glutamate release (FIG. 22A), hyperfunction of NMDAR (FIG. 22B) and LDH levels (FIG. 22C) in cortical cells and apoptosis in neurons and astrocytes (FIGS. 22D and 22E), suggesting these compounds have a protective effect on OGDR-induced cellular injury and apoptosis.

FIG. 23 shows the lactate dehydrogenase (LDH) level in WT cortical cells treated with or without sorafenib (10 μM), erastin (10 μM) or sulfasalazine (SAS, 500 μM) at various time points after oxygen glucose deprivation (OGD). The start of sorafenib, erastin or sulfasalazine exposure was delayed to 2-24 h after OGD. The LDH assay was carried out at 24 hours after reperfusion. *P<0.01 compared to drugs-untreated cells (Un), one-way ANOVA. Error bars denote the standard deviation among triplicate experiments.

As described above, lactate dehydrogenase (LDH) is a marker for cell apoptosis. In FIG. 23, compared to the drugs-untreated cells (Un), sorafenib, erastin or sulfasalazine significantly reduced the OGD-induced increases in LDH level in time-dependent manner. LDH level was significantly reduced by 30%-80% at 0-12 h post-treatment with sorafenib, erastin or sulfasalazine after OGD. Therefore, these compounds has a neuroprotective effect within 12 h after OGDR, suggesting the inhibitors of system x_{c}⁺ are able to prolong therapeutic window for ischemic brain damage.

Genetic Deficiency of System x_{c}⁻ Reduces Cerebral Ischemia-Induced Glutamate Efflux, Hyperfunction of NMDAR and Infarct Size

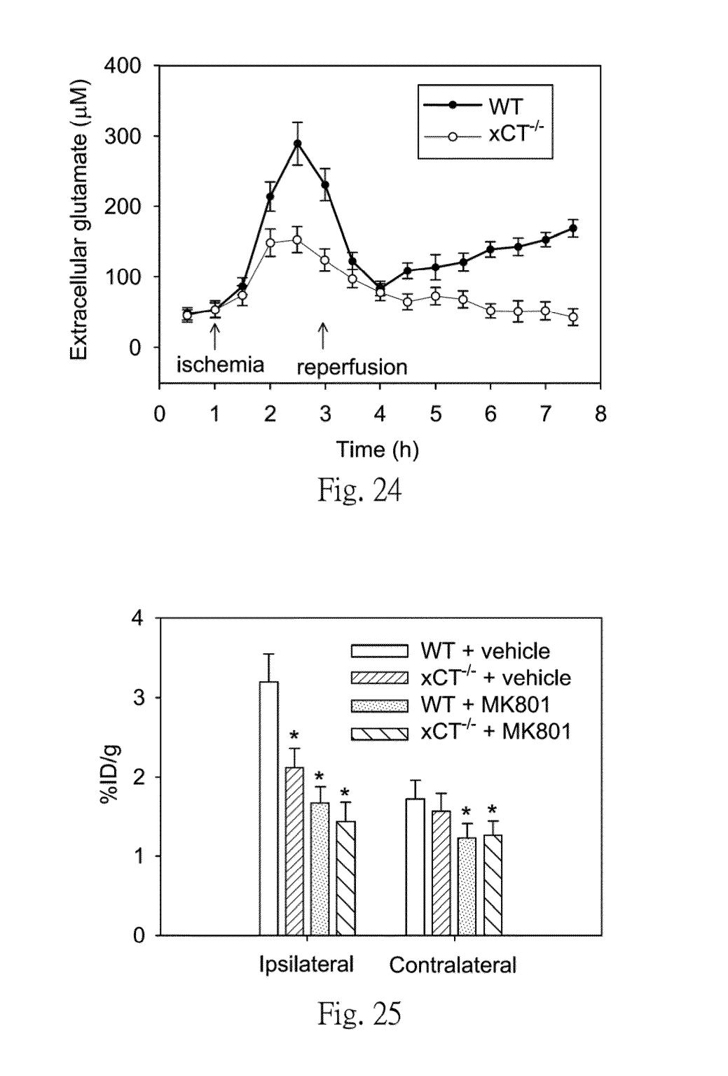

In order to extend these in vitro findings to an in vivo system, xCT^{-/-} and wild type mice received CIR. A microdialysis assay of glutamate concentration in both xCT^{-/-} and wild type mice was performed. FIG. 24 is a diagram showing the kinetics of extracellular glutamate content in ischemic cortex from wild type (WT) and xCT^{-/-} mice with cerebral ischemia/reperfusion (CIR). Time points for cerebral ischemia and reperfusion are indicated. FIG. 24 shows that CIR resulted in an immediate increase in extracellular glutamate level, although less so in xCT^{-/-} mice. The glutamate efflux of the xCT^{-/-} mice peaked 90 min later and decreased thereafter although not to pre-ischemic levels. Interestingly, a second, gradual increase in glutamate levels occurred 1 h after reperfusion in wild type but not in xCT^{-/-} mice.

To determine in vivo activation of NMDAR, mice were injected with ¹⁸F-FSAG at 12 h after reperfusion for ex vivo biodistribution studies and positron emission tomography (PET) imaging studies. FIG. 25 shows the accumulation of ¹⁸F-labelled alkylthiophenyl guanidine in the ipsilateral and contralateral cerebral hemispheres from WT and xCT^{-/-} mice with CIR at 12 h after reperfusion. *P is <0.01 compared to WT with vehicle, Student's t-test. Ex vivo biodistribution studies in brain tissues showed that the binding radioactivity of ¹⁸F-FSAG to NMDAR was significantly increased in the ipsilateral cerebral hemisphere compared with the contralateral cerebral hemisphere. There was a significant decrease in the binding of ¹⁸F-FSAG to NMDAR in xCT^{-/-} mice as compared to wild type mice. Additionally, wild type and xCT^{-/-} mice treated with MK801 showed a significant reduction in ¹⁸F-FSAG accumulation in both the ipsilateral and contralateral hemispheres as compared to mice with vehicle. This result indicated the radioligand specificity for the visualisation of NMDAR activation in vivo.

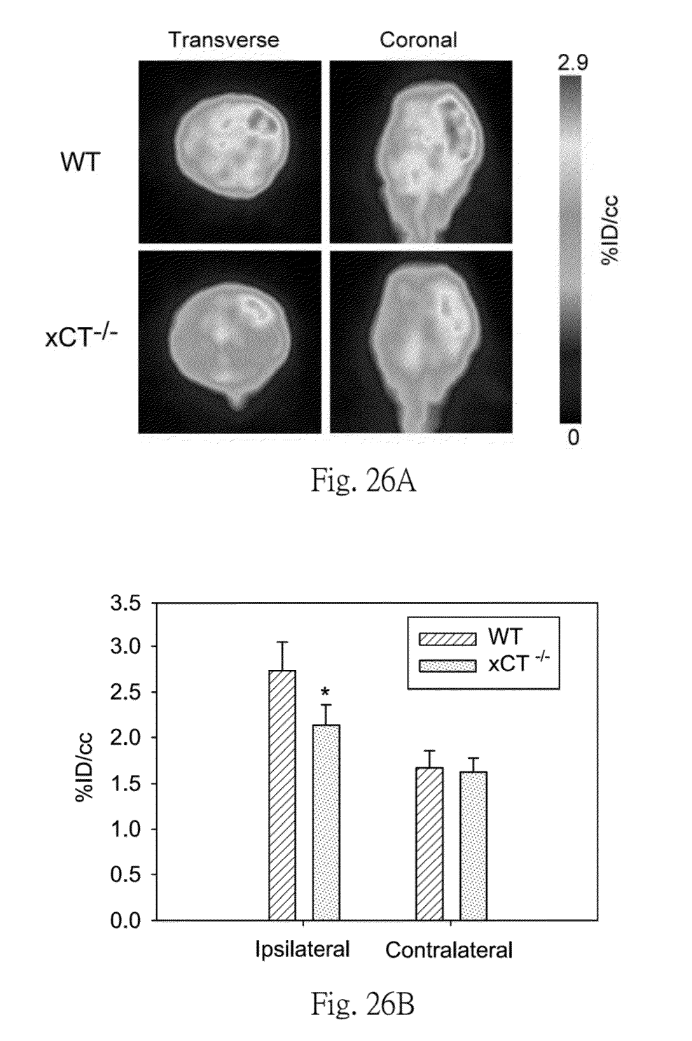

FIG. 26A shows ¹⁸F-FSAG PET imaging of brains in WT and xCT^{-/-} mice with CIR at 12 h after reperfusion; FIG. 26B shows accumulation of ¹⁸F-FSAG in the ipsilateral and contralateral cerebral hemispheres of WT and xCT^{-/-} mice with CIR 12 h reperfusion. *P is <0.01 compared to WT, Student's t-test. The vertical axial unit, % ID/cc, means the percentage injected dose per gram tissue. PET imaging also demonstrated that xCT^{-/-} mice exhibited an appreciably lower accumulation of radioactive substances in the ipsilateral hemisphere compared with wild type mice, suggesting that genetic deficiency of system x_{c}⁻ decreases CIR-mediated hyperfunction of NMDAR.

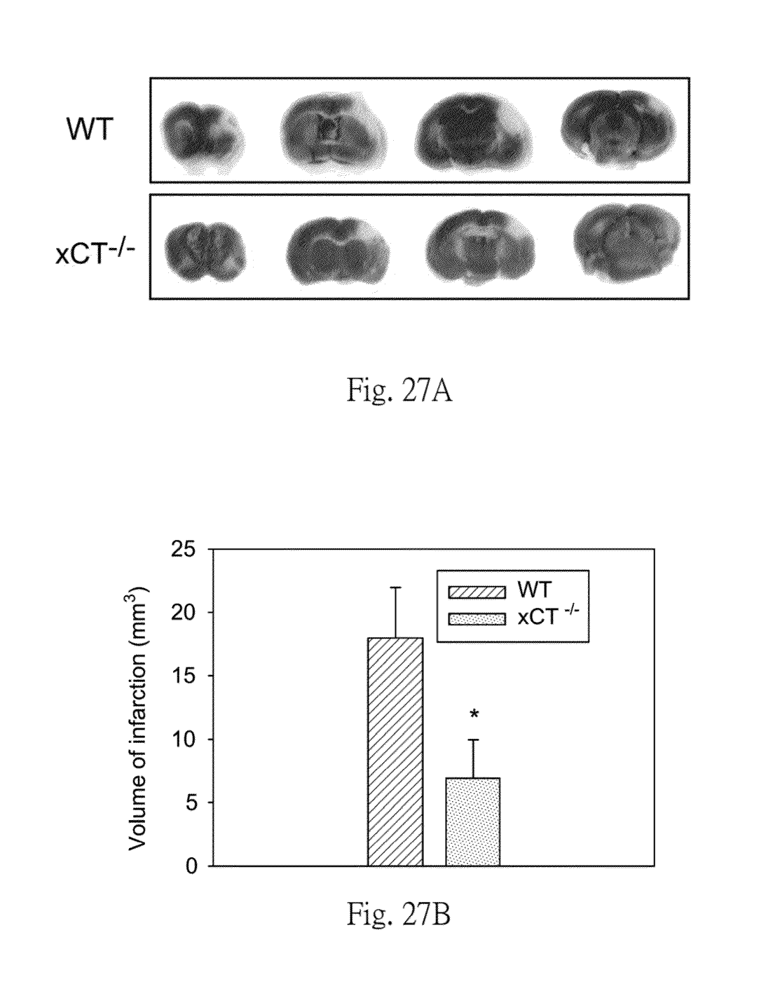

Finally, 2,3,5-triphenyltetrazolium chloride (TTC) staining assay for differentiating living cells (stained to red colour) and death cells (white colour) was performed. FIGS. 27A and 27B respectively show representative photographs of 2,3,5-triphenyltetrazoliu chloride (TTC) staining and calculated infarct volume in brains from WT and xCT^{-/-} mice with CIR 3 days post-reperfusion. *P is <0.01 compared to WT, Student's t-test. The numbers of both WT and xCT^{-/-} animals were 6. The results demonstrated that cortical infarct volume (death cells) was significantly smaller in xCT^{-/-} mice as compared to wild type mice. Thus, genetic deficiency of system x_{c}⁻ decreased infarct volume of the ischemic brains.

Pharmacological Inhibition of System x_{c}⁻ Reduces Cerebral Ischemia-Induced Glutamate Excitotoxicity

To explore the therapeutic potential of manipulation of system x_{c}⁻ following stroke, a drug that blocks system x_{c}⁻ (Chung W J, Lyons S A, Nelson G M, Hamza H, Gladson C L, Gillespie G Y, et al. Inhibition of cystine uptake disrupts the growth of primary brain tumors. The Journal of neuroscience: the official journal of the Society for Neuroscience. 2005; 25(31):7101-10; Buckingham S C, Campbell S L, Haas B R, Montana V, Robel S, Ogunrinu T, et al. Glutamate release by primary brain tumors induces epileptic activity. Nature medicine. 2011; 17(10):1269-74) was used to treat mice exposed to CIR. The drug used here was SAS, which is a drug approved by the US Food and Drug Administration (FDA).

SAS is formed by combining 5-ASA with SP by an azo bond, the disruption of which abolishes the inhibition of system x_{c}⁻ (Shukla K, Thomas A G, Ferraris D V, Hin N, Sattler R, Alt J, et al. Inhibition of xc(−) transporter-mediated cystine uptake by sulfasalazine analogs. Bioorganic & medicinal chemistry letters. 2011; 21(20):6184-7). Therefore, a mixture of 5-ASA and SP as a control was used to rule out other potential effects from 5-ASA and SP.

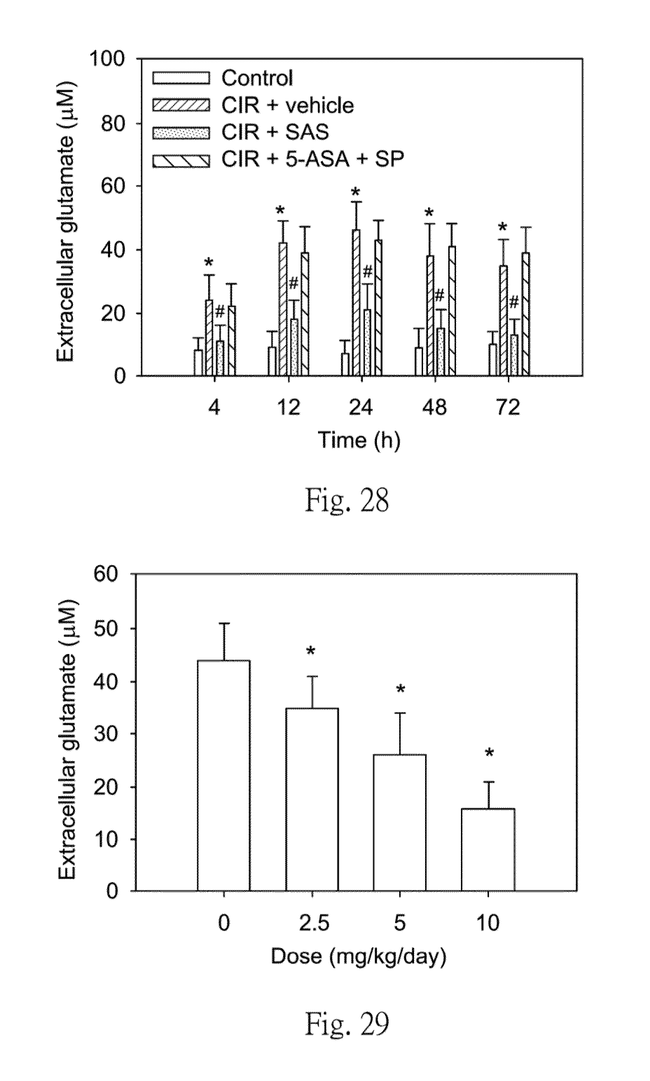

FIG. 28 shows extracellular glutamate content in ischemic cortex from rats with cerebral is hemia/reperfusion (CIR) followed by sulfasalazine (SAS) or the mixture of sulfapyridine (5-ASA) and salicylate (SP) as a control at 4 to 72 h after reperfusion. Mice were treated with vehicle, SAS (5 mg/kg/day, BID) as well as the mixture of 5-ASA (3.12 mg/kg/day, BID) and SP (1.72 mg/kg, BID) for 3 days. *P is <0.001 compared to control without CIR, one-way ANOVA. ^{#}P is <0.001 compared to CIR with vehicle, one-way ANOVA. Compared to the vehicle or the mixture of 5-ASA and SP-treated animals in FIG. 28, animals treated with SAS showed a reduction in extracellular glutamate content at 4 to 72 h after reperfusion.

FIG. 29 is a diagram showing suppression of glutamate efflux in ischemic cortex from rats with CIR followed by SAS at different dosages. *P is <0.05 compared to control without SAS, one-way ANOVA. In FIG. 29, it was observed that SAS also inhibited CIR-induced glutamate release in a dose-dependent manner.

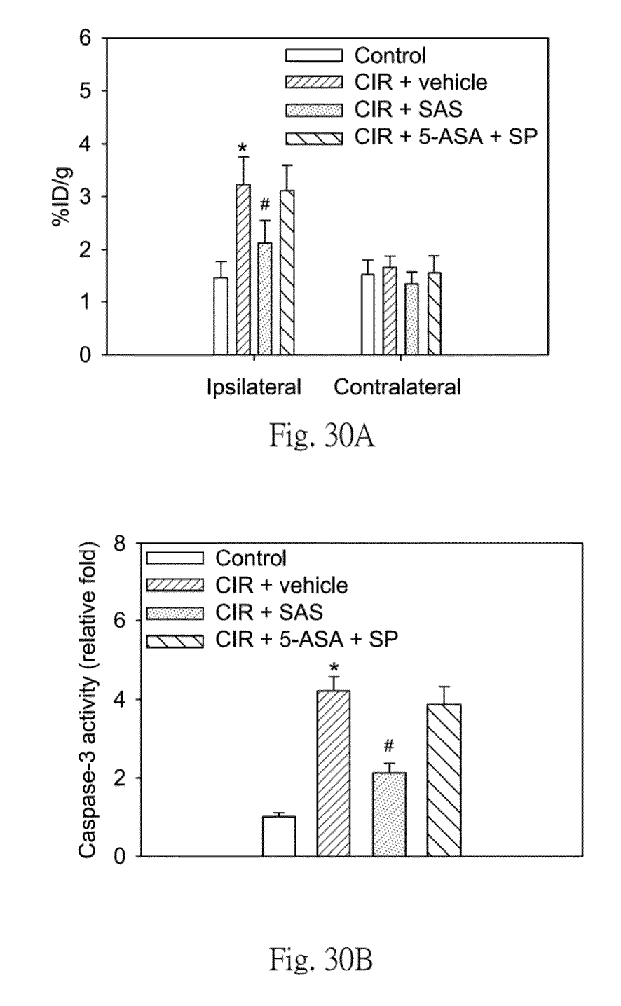

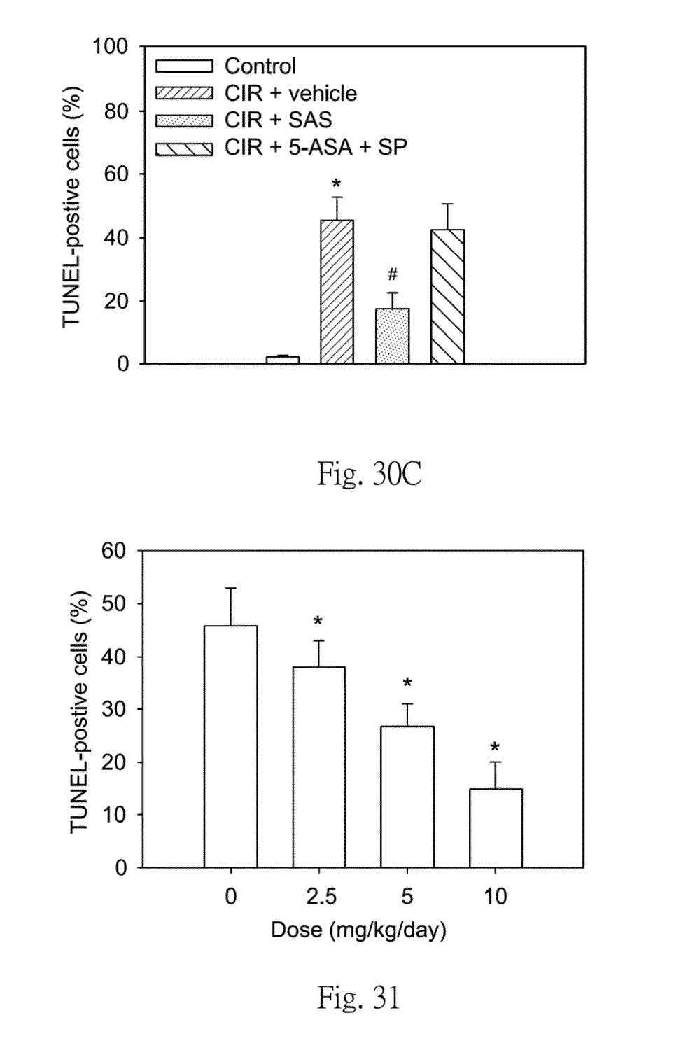

FIG. 30A is a diagram showing ¹⁸F-FSAG accumulation in the ipsilateral and contralateral cerebral hemispheres in the ipsilateral cerebral hemispheres of rats with CIR followed by SAS or the mixture of the mixture of 5-ASA and SP at 24 h after reperfusion. *P is <0.001 compared to control without CIR, Student's t-test. ^{#}P is <0.001 compared to CIR with vehicle, Student's t-test. From FIG. 30A, it was observed that the accumulation of ¹⁸F-FSAG in the ipsilateral cerebral hemisphere was significantly lower in animals with SAS treatment compared to control animals with vehicle or a mixture of 5-ASA and SP.

FIG. 30B is a diagram showing caspase 3 activity in the ipsilateral cerebral hemispheres of rats with CIR followed by SAS or the mixture of the mixture of 5-ASA and SP at 24 h after reperfusion. *P is <0.001 compared to control without CIR, Student's t-test. ^{#}P is <0.001 compared to CIR with vehicle, Student's t-test. From FIG. 30B, it was observed that the activity of caspase 3 in the ipsilateral cerebral hemisphere was significantly lower in animals with SAS treatment compared to control animals with vehicle or a mixture of 5-ASA and SP.

FIG. 30C is a diagram showing TUNEL-positive cells in the ipsilateral cerebral hemispheres of rats with CIR followed by SAS or the mixture of the mixture of 5-ASA and SP at 24 h after reperfusion. *P is <0.001 compared to control without CIR, Student's t-test. ^{#}P is <0.001 compared to CIR with vehicle, Student's t-test. Terminal deoxynucleotidyl transferase dUTP nick end labeling (TUNEL) is a method for detecting DNA fragmentation that results from apoptotic signaling cascades, by labeling the terminal end of nucleic acids. From FIG. 30C, it was observed that TUNEL staining in the ischemic penumbra of animals showed that SAS treatment significantly reduced the number of TUNEL-positive cells (i.e. apoptotic cells) 24 h after reperfusion compared with vehicle or mixture of 5-ASA and SP treatment.

FIG. 31 is a diagram showing inhibition of TUNEL-positive cells in the ipsilateral cerebral hemisphere of rats with CIR followed by SAS with different dosages. *P is <0.05 compared to control without SAS, one-way ANOVA. Number of animals was 6-8. From FIG. 31, it was observed that SAS also decreased CIR-induced apoptosis in a dose-dependent manner.

Taken together, these results indicate that the pharmacological inhibition of system x_{c}⁻ by SAS decreased CIR-induced glutamate release, hyperfunction of NMDAR, and apoptosis.

Pharmacological Inhibition of System x_{c}⁻ Reduces Infarct Volume and Improves Neurological Behavior after Cerebral Ischemia

Whether pharmacological inhibition of system x_{c}⁻ has a therapeutic outcome in CIR was determined. MRI was utilised to non-invasively observe the volume of cerebral infarction in cerebral ischemic rats with or without SAS treatment.

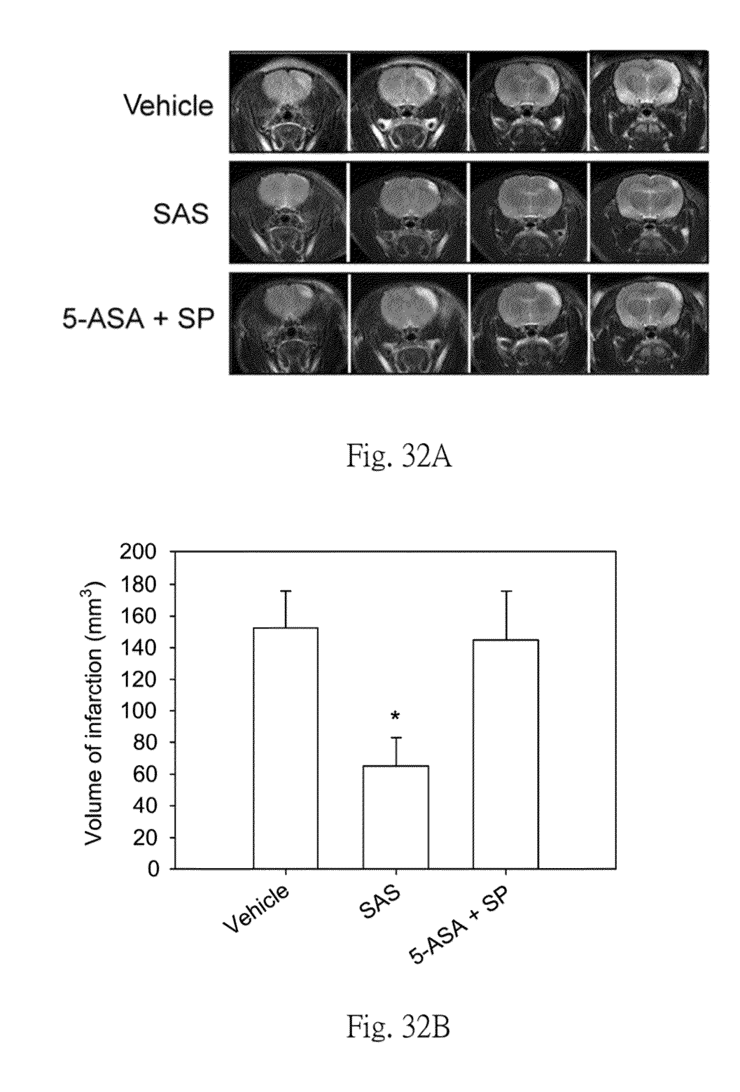

FIGS. 32A and 32B respectively shows representative images of magnetic resonance images and calculated infarct volume in brains from rats with cerebral ischemia/reperfusion (CIR) followed by sulfasalazine (SAS) or the mixture of sulfapyridine (5-ASA) and salicylate (SP) on day 28 after reperfusion. In FIG. 32B, *P is <0.001 compared to vehicle, Student's t-test. MRIs showed that cortical infarcts in rats treated with SAS have remarkable size reductions on day 28. By contrast, cortical infarcts in control rats with vehicle or mixture of 5-ASA and SP treatment showed only a small decrease in infarct size.

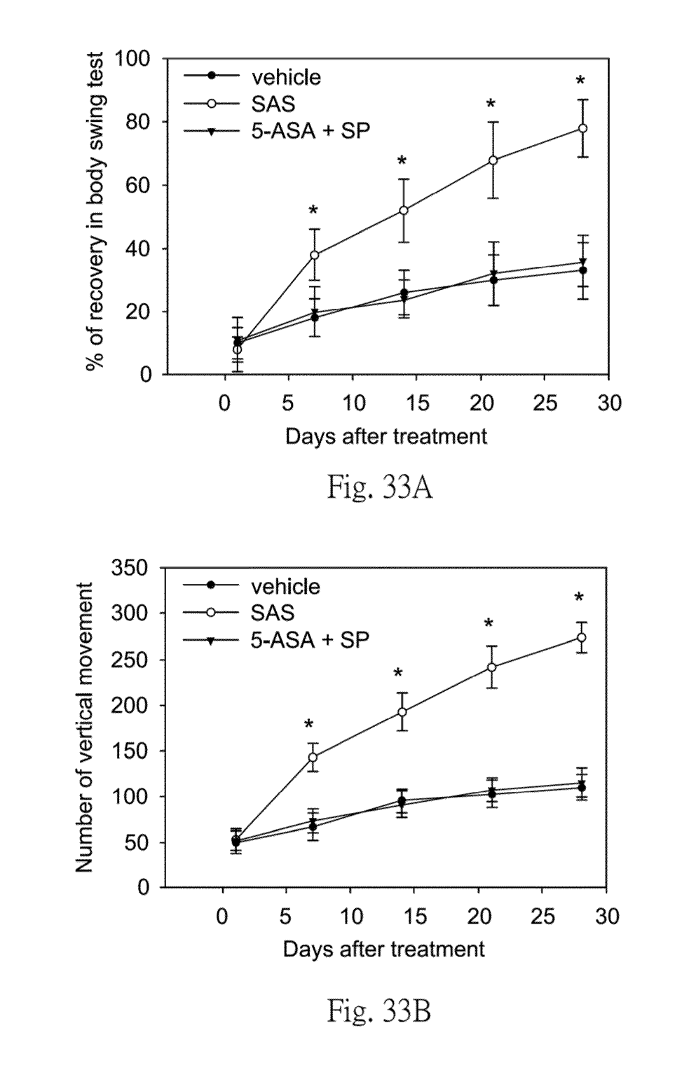

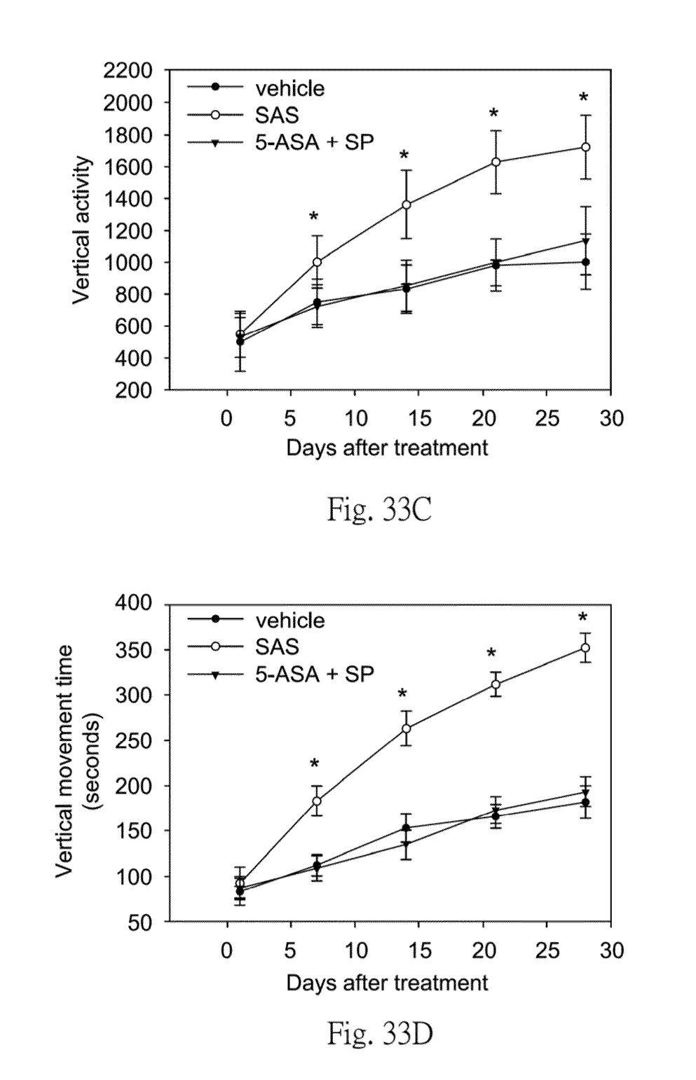

To evaluate the neuroprotective effect of SAS treatment during CIR, body asymmetry trials and locomotor activity tests were used to assess neurological behaviour in SAS-treated and control stroke rats with vehicle or mixture of 5-ASA and SP treatment. FIGS. 33A-330 are diagrams respectively showing body asymmetry, number of vertical movement, vertical activity, and vertical movement time in rats with CIR followed by SAS or the mixture of 5-ASA and SP on days 7, 14, 21, 28 after reperfusion. *P is <0.05 compared to vehicle, one-way ANOVA.

As seen in FIG. 33A, from days 7 to 28 after treatment, rats treated with SAS exhibited significantly reduced body asymmetry compared with control rats. In FIGS. 33B to 33D, locomotor activity (e.g. number of vertical movements, vertical activity, and vertical movement time) was significantly higher in rats that received SAS treatment compared with controls between 7 and 28 days after CIR.

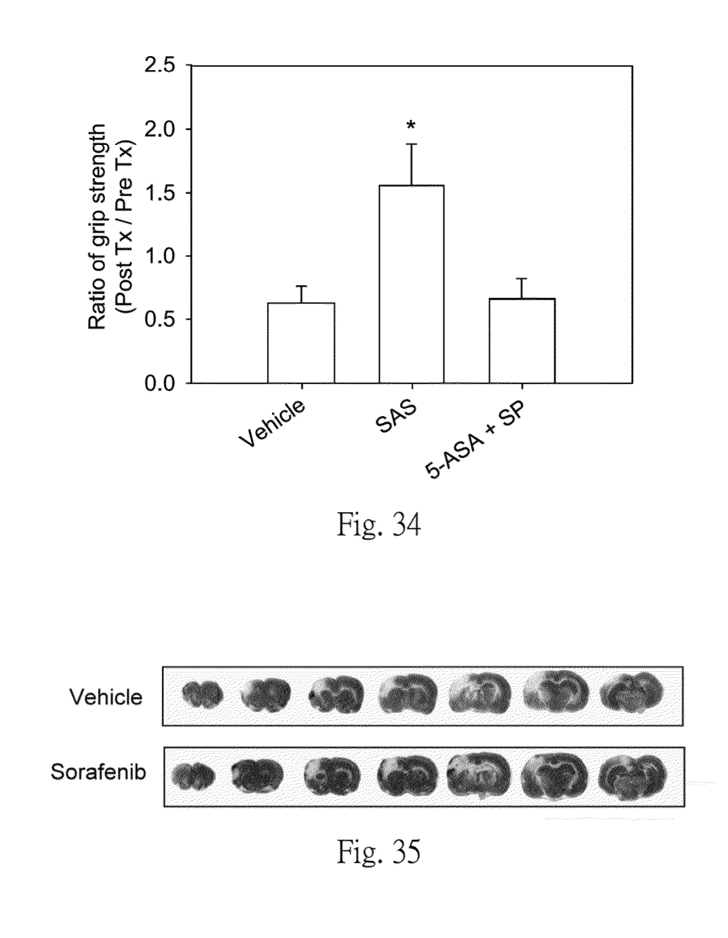

Grip strength was measured before treatment and at a days after each of the 2 treatments in order to examine changes in forelimb strength in all experimental rats. FIG. 34 is a diagram showing grip strength ratio in rats with CIR followed by SAS or the mixture of 5-ASA and SP on day 28 after reperfusion. *P is <0.01 compared to vehicle, Student's t-test. All of rats were treated with vehicle, SAS (5 mg/kg/day, BID) and the mixture of 5-ASA (3.12 mg/kg/day, BID) and SP (1.72 mg/kg, BID) for 3 days. Number of animals of each group was 6-8. As observed in FIG. 33, a higher percentage of improvement in grip strength was found in the SAS-treated group compared with the control groups.

To test whether the other system x_{c}⁻ inhibitor also have therapeutic benefits for animals with CIR. 2,3,5-Triphenyltetrazolium chloride (TTC) staining assay was utilized to determine the cortical infarct volume in rats with CIR followed by vehicle or sorafenib for 3 days. FIG. 35 is a diagram showing representative photographs of TTC staining (g) in brains from rats with cerebral ischemia/reperfusion (CIR) followed by vehicle or sorafenib (30 mg/kg ip) for 3 days. *P is <0.001 compared to vehicle. Number of animals in each group was 6. In FIG. 35, TTC staining assay demonstrated that cortical infarct volume was significantly smaller in rats treated with sorafenib as compared to control rats with vehicle. Collectively, these results suggest that pharmacological inhibition of system via SAS or other system x_{c}⁻ inhibitor provides therapeutic benefits for animals with CIR.

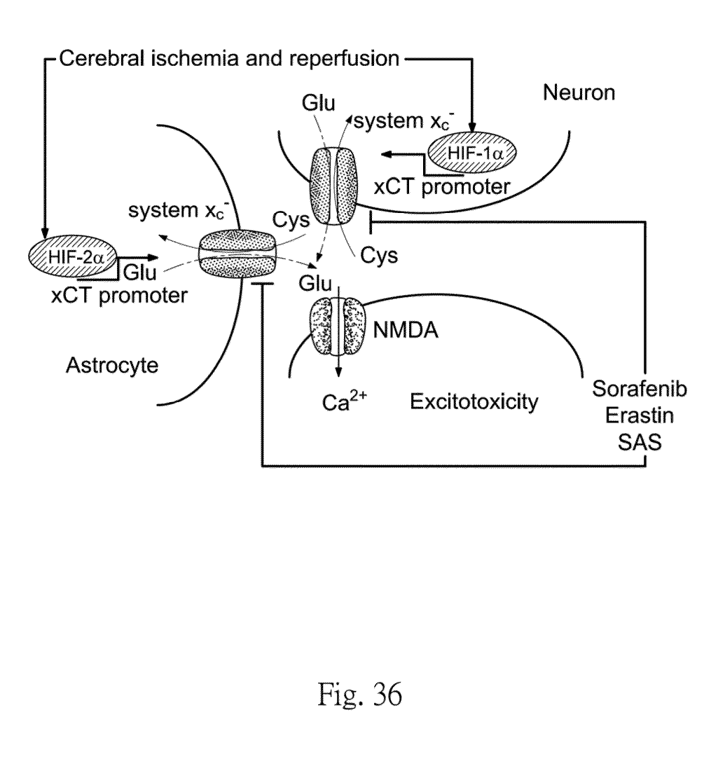

Accordingly, this disclosure provides strong evidence that system x_{c}⁻ promotes the dual phase of CIR-induced elevation of extracellular glutamate and contributes to the excessive activation of NMDAR and excitotoxicity in brain. In the foregoing, the disclosure also provides a novel aspect that HIF-1α and HIF-2α transactivation of xCT expression is required for OGDR or CIR-induced glutamate release and excitotoxicity. On the basis of the foregoing findings, a model is proposed in FIG. 36. FIG. 36 is a diagram showing a working model of HIF-1-regulated system x_{c}⁻ in CIR-mediated imbalance of glutamate homeostasis and excitotoxicity and its therapeutic innervation.

In FIG. 36, brain ischemia and reperfusion increases HIF-1α or HIF-2α accumulation in neurons and astrocytes by promoting protein synthesis or inhibiting protein degradation. The cytoplasmic HIF-1α or HIF-2α then translocates to the nucleus, recognises a cognate sequence on the xCT promoter, induces xCT expression, and promotes long-term system x_{c}⁻ function and glutamate excitotoxicity. The blockade of system x_{c}⁻ by the selective inhibitors sorafenib, erastin or SAS inhibited the dual phases of glutamate excitotoxicity and prevented neural and astrocyte injuries or death during CIR.

Both the HIF-1α and HIF-2α proteins are present in cortical neurons and astrocytes. HIF-1α protein expression is more prominent in neurons, whereas HIF-2α protein levels are higher in astrocytes. This discrepancy might be related to the developmental stage of the cultured neurons. HIF-1 contributes to a robust and long-lasting CIR-triggered xCT expression and system function. Therefore, a novel concept that HIF-1 plays a role in regulating glutamate homeostasis via system x_{c}⁻ in response to cerebral hypoxia or ischemia is provided.

The results also demonstrated a dual phase of CIR-induced the elevation of glutamate in ischemic temporal cortex in wild type mice. Most importantly, our results indicated that genetic deficiency of system x_{c}⁻ in xCT^{-/-} mice showed not only a dramatic decrease in early phase ischemic-induced elevated glutamate levels but also an inhibition of late phase perfusion-mediated glutamate release, suggesting that system x_{c}^{-/-} is critical mediator in the dual phase of CIR-induced glutamate release and excitotoxicity.

Here, it was found that the expression of xCT and the function of system x_{c}⁻ rapidly increased in response to CIR, and this effect continued for 7 days in ischemic brain tissues. Moreover, genetic deficiency of system decreased CIR-mediated elevation of glutamate levels, hyperfunction of the NMDAR and in vivo infarct volume, suggesting a critical role of system x_{c}⁻ in CIR-mediated glutamate release and consequent glutamate-induced neuronal excitotoxicity in our ischemic stroke model.

In addition, the results derived from rodent brain slices in response to oxygen and glucose deprivation indicated system x_{c}⁻ played a role in oxygen and glucose deprivation-mediated elevation of the extracellular glutamate concentration, overactivation of extrasynaptic NMDARs, and ischemic-induced neuronal death. In light of these findings, it is intriguing to postulate that system x_{c}⁻ mediated excitotoxicity might contribute to early and late phase events of CIR-induced ischemic damage.

The data presented here demonstrate that system is a promising therapeutic target for stroke. Pharmacological inhibition of system x_{c}⁻ with administration of sorafenib, regorafenib, erastin or SAS significantly inhibited OGDR-induced cellular injury and apoptosis in neurons and astrocytes. Moreover, animals with CIR that were administered SAS had significant therapeutic benefits including reduction of infarct volume and improvement of neurological behavior, suggesting inhibition of system x_{c}⁻for the prevention of stroke-induced neurotoxicity.

Accordingly, it can be predicted that these treatments might extend the therapeutic time window, compared to thrombolytic therapy, as system x_{c}⁻-mediated glutamate excitotoxicity can last for up to 7 days. Furthermore, given that these compounds are already characterised and FDA-approved, using SAS, sorafenib or regorafenib for neuroprotection following stroke potentially represents a less expensive and expedient option in the clinical setting.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A method of treating oxygen glucose deprivation/re-oxygenation (OGDR)-induced cellular injury and apoptosis in neurons and astrocytes of a higher vertebrate animal, comprising:
administering an effective amount of an inhibitor of cysteine-glutamate transporter (i.e. system x_{c}⁻) in the higher vertebrate animal to decrease a concentration of extracellular glutamate in the neurons and the astrocytes to treat the OGDR-induced cellular injury and apoptosis in the neurons and the astrocytes.

2. The method of claim 1, wherein the inhibitor comprises sorafenib or its derivative, regorafenib, which does not need to be administered with tissue-type plasminogen activator (abbreviated as tPA).

3. The method of claim 1, wherein the inhibitor comprises erastin or sulfasalazine.

4. The method of claim 1, wherein the higher vertebrate animal is a mammal.

5. The method of claim 1, wherein the higher vertebrate animal is a human.

6. A method of reducing cortical infarct volume in a brain of a higher vertebrate animal suffering ischemic or hypoxia brain injury, comprising:
administering an effective amount of an inhibitor of cysteine-glutamate transporter (i.e. system x_{c}⁻) in the higher vertebrate animal, so that the cortical infarct volume in the brain is reduced.

7. The method of claim 6, wherein the inhibitor comprises sorafenib or its derivative, regorafenib, which does not need to be administered with tissue-type plasminogen activator (abbreviated as tPA).

8. The method of claim 6, wherein the inhibitor comprises erastin or sulfasalazine.

9. The method of claim 6, wherein the higher vertebrate animal is a mammal.

10. The method of claim 6, wherein the higher vertebrate animal is a human.

11. A method of reducing cerebral ischemia and reperfusion (CIR)-induced glutamate release as well as excitotoxicity to central nervous system (CNS), comprising:
administering an effective amount of an inhibitor of cysteine-glutamate transporter (i.e. system x_{c}⁻) in the higher vertebrate animal to decrease a concentration of extracellular glutamate, so that the CIR-induced glutamate release as well as excitotoxicity to CNS is reduced.

12. The method of claim 11, wherein the inhibitor comprises sorafenib or its derivative, regorafenib, which does not need to be administered with tissue-type plasminogen activator (abbreviated as tPA).

13. The method of claim 11, wherein the inhibitor comprises erastin or sulfasalazine.

14. The method of claim 11, wherein the higher vertebrate animal is a mammal.

15. The method of claim 11, wherein the higher vertebrate animal is a human.

16. A method of treating ischemic brain damage, the method comprising:
administering an effective amount of an inhibitor of cysteine-glutamate transporter (i.e. system x_{c}⁻) in the higher vertebrate animal within 12 hours after the occurring of oxygen glucose deprivation.

17. The method of claim 16, wherein the inhibitor comprises sorafenib or its derivative, regorafenib, which does not need to be administered with tissue-type plasminogen activator (abbreviated as tPA).

18. The method of claim 16, wherein the inhibitor comprises erastin or sulfasalazine.

19. The method of claim 16, wherein the higher vertebrate animal is a mammal.

20. The method of claim 16, wherein the higher vertebrate animal is a human.
